SEQUENCE LISTING (28 sequences)

SEQ ID NO: 1 - protein, 6 aa, Artificial Sequence
  Synthetic
TNYWMN

SEQ ID NO: 2 - protein, 17 aa, Artificial Sequence
  Synthetic
MIHPSDSETRLNQKFQG

SEQ ID NO: 3 - protein, 17 aa, Artificial Sequence
  Synthetic
MIHPSDSETRLNQKLQG

SEQ ID NO: 4 - protein, 13 aa, Artificial Sequence
  Synthetic
EGRLGLRSYAMDY

SEQ ID NO: 5 - protein, 17 aa, Artificial Sequence
  Synthetic
RSSQSLLNSSNQKNYLA

SEQ ID NO: 6 - protein, 7 aa, Artificial Sequence
  Synthetic
FASTRES

SEQ ID NO: 7 - protein, 9 aa, Artificial Sequence
  Synthetic
QQHYITPLT

SEQ ID NO: 8 - protein, 114 aa, Artificial Sequence
  Synthetic
DIQMTQSPSSLSASVGDRVTITCRSSQSLLNSSNQKNYLAWYQQKPGKVPKLLIYFASTRESGVPSRFSGSGSGTDFTLTISSLQPEDVATYFCQQHYITPLTFGGGTKVEIKR

SEQ ID NO: 9 - protein, 242 aa, Artificial Sequence
  Synthetic
MDMRVPAQLLGLLLLWLRGARCDIQMTQSPSSLSASVGDRVTITCRSSQSLLNSSNQKNYLAWYQQKPGKVPKLLIYFASTRESGVPSRFSGSGSGTDFTLTISSLQPEDVATYFCQQHYITPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 122 aa, Artificial Sequence
  Synthetic
QVQLVQSGAEVKKPGSSVKVSCKASGYTFTNYWMNWVRQAPGQGLEWMGMIHPSDSETRLNQKFQGRVTITVDKSTSTAYMELSSLRSEDTAVYYCAREGRLGLRSYAMDYWGQGTLVTVSS

SEQ ID NO: 11 - protein, 471 aa, Artificial Sequence
  Synthetic
MDMRVPAQLLGLLLLWLRGARCQVQLVQSGAEVKKPGSSVKVSCKASGYTFTNYWMNWVRQAPGQGLEWMGMIHPSDSETRLNQKFQGRVTITVDKSTSTAYMELSSLRSEDTAVYYCAREGRLGLRSYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 114 aa, Artificial Sequence
  Synthetic
DIQMTQSPSSLSASVGDRVTITCRSSQSLLNSSNQKNYLAWYQQKPGKAPKLLIYFASTRESGVPSRFSGSGSGTDFTLTISSLQPEDFATYFCQQHYITPLTFGGGTKVEIKR

SEQ ID NO: 13 - protein, 242 aa, Artificial Sequence
  Synthetic
MDMRVPAQLLGLLLLWLRGARCDIQMTQSPSSLSASVGDRVTITCRSSQSLLNSSNQKNYLAWYQQKPGKAPKLLIYFASTRESGVPSRFSGSGSGTDFTLTISSLQPEDFATYFCQQHYITPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, Artificial Sequence
  Synthetic
QVQLVQSGAEVKKPGASVKVSCKASGYTFTNYWMNWVRQAPGQGLEWMGMIHPSDSETRLNQKLQGRVTITVDKSTSTAYMELRSLRSDDTAVYYCAREGRLGLRSYAMDYWGQGTLVTVSS

SEQ ID NO: 15 - protein, 471 aa, Artificial Sequence
  Synthetic
MDMRVPAQLLGLLLLWLRGARCQVQLVQSGAEVKKPGASVKVSCKASGYTFTNYWMNWVRQAPGQGLEWMGMIHPSDSETRLNQKLQGRVTITVDKSTSTAYMELRSLRSDDTAVYYCAREGRLGLRSYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 17 aa, Artificial Sequence
  Synthetic
MIHPSDSETRLNQKFKD

SEQ ID NO: 17 - protein, 17 aa, Artificial Sequence
  Synthetic
KSSQSLLQSSNQKNYLA

SEQ ID NO: 18 - protein, 17 aa, Artificial Sequence
  Synthetic
KSSQSLLNSSNQKNYLA

SEQ ID NO: 19 - protein, 115 aa, Artificial Sequence
  Synthetic
GAELVRPGASVKLSCKASGYSFTNYWMNWVKQRPGQGLEWIGMIHPSDSETRLNQKFKDKATLTVDKSSSTAYMQLSSPTSEDSAVYYCAREGRLGLRSYAMDYWGQGTSVTVSS

SEQ ID NO: 20 - protein, 104 aa, Artificial Sequence
  Synthetic
PSSLAMSVGQKVTMSCKSSQSLLNSSNQKNYLAWYQQKPGQSPKLLVYFASTRESGVPDRFVGSGSGTDFTLTITSVQAEDLADYFCQQHYITPLTFGAGTKLE

SEQ ID NO: 21 - protein, 122 aa, Artificial Sequence
  Synthetic
QVQLVQSGSELKKPGASVKVSCKASGYTFTNYWMNWVRQAPGQGLEWMGMIHPSDSETRLNQKFKDRVTITVDKSTSTAYMELSSLRSEDTAVYYCAREGRLGLRSYAMDYWGQGTLVTVSS

SEQ ID NO: 22 - protein, 114 aa, Artificial Sequence
  Synthetic
DIVMTQSPDSLAVSLGERATINCKSSQSLLNSSNQKNYLAWYQQKPGQPPKLLVYFASTRESGVPDRFSGSGSGTDFTLTISSLQAEDVAVYFCQQHYITPLTFGGGTKVEIKR

SEQ ID NO: 23 - protein, 471 aa, Artificial Sequence
  Synthetic
MEWSWVFLFFLSVTTGVHSQVQLVQSGSELKKPGASVKVSCKASGYTFTNYWMNWVRQAPGQGLEWMGMIHPSDSETRLNQKFKDRVTITVDKSTSTAYMELSSLRSEDTAVYYCAREGRLGLRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 242 aa, Artificial Sequence
  Synthetic
MDMRVPAQLLGLLLLWLRGARCDIVMTQSPDSLAVSLGERATINCKSSQSLLNSSNQKNYLAWYQQKPGQPPKLLVYFASTRESGVPDRFSGSGSGTDFTLTISSLQAEDVAVYFCQQHYITPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 122 aa, Artificial Sequence
  Synthetic
QVQLVQSGAELKKPGASVKVSCKASGYTFTNYWMNWVRQAPGQGLEWMGMIHPSDSETRLNQKFKDRVTITVDKSTSTAYMELSSLRSEDTAVYYCAREGRLGLRSYAMDYWGQGTLVTVSS

SEQ ID NO: 26 - protein, 114 aa, Artificial Sequence
  Synthetic
DIVMTQSPDSLAVSLGERATINCKSSQSLLQSSNQKNYLAWYQQKPGQPPKLLVYFASTRESGVPDRFSGSGSGTDFTLTISSLQAEDVAVYFCQQHYITPLTFGGGTKVEIKR

SEQ ID NO: 27 - protein, 471 aa, Artificial Sequence
  Synthetic
MDMRVPAQLLGLLLLWLRGARCQVQLVQSGAELKKPGASVKVSCKASGYTFTNYWMNWVRQAPGQGLEWMGMIHPSDSETRLNQKFKDRVTITVDKSTSTAYMELSSLRSEDTAVYYCAREGRLGLRSYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 242 aa, Artificial Sequence
  Synthetic
MDMRVPAQLLGLLLLWLRGARCDIVMTQSPDSLAVSLGERATINCKSSQSLLQSSNQKNYLAWYQQKPGQPPKLLVYFASTRESGVPDRFSGSGSGTDFTLTISSLQAEDVAVYFCQQHYITPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0406915 A1
(43) Pub. Date: Dec. 21, 2023

(54) ANTI-DKK2 ANTIBODY, COMPOSITION CONTAINING ANTI-DKK2 ANTIBODY, AND USE THEREOF

(71) Applicant: HJB (HANGZHOU) CO., LTD.), Hangzhou, Zhejiang (CN)

(72) Inventors: Dayou Liu, Jianggan District Hangzhou, Zhejiang (CN); Christopher Hwang, Jianggan District Hangzhou, Zhejiang (CN)

(21) Appl. No.: 18/255,567

(22) PCT Filed: Nov. 16, 2021
(86) PCT No.: PCT/CN2021/130835

§ 371 Date: Jun. 1, 2023

(30) Foreign Application Priority Data
Dec. 2, 2020 (CN) 202011402397.9

(51) Int. Cl. C07K 16/18; A61P 35/00

(52) U.S. Cl. CPC C07K 16/18; A61P 35/00; C07K 2317/565

(57) ABSTRACT

The present disclosure relates to a humanized and CMC properties-improved anti-DKK2 antibody, a composition containing the antibody, and a method for treating diseases using the antibody or the composition.

### REFERENCE TO AN ELECTRONIC SEQUENCE LISTING

The contents of the electronic sequence listing (880175_401USPC_SeqListing.txt; Size: 38,714 bytes; and Date of Creation: May 31, 2023) is herein incorporated by reference in its entirety.

### TECHNICAL FIELD

The present disclosure relates to a humanized and CMC (chemical, manufacturing and control) properties-improved anti-DKK2 antibody, a composition containing the antibody, and a method for treating diseases using the antibody or the composition.

### BACKGROUND ART

The part of statements merely provides background information related to the present disclosure and do not necessarily constitute the prior art.

Cancer is a major health problem worldwide. Each year, tens of millions of people are diagnosed with cancer around the world, and more than half of the patients eventually die from it. About one-half of all men and one-third of all women in the US will be diagnosed with a cancer at some point during their lifetime, and one in four deaths is caused by cancer (Jemal et al., CA Cancer J. Clin., 2002, 52:23-47; Howlader et al., SEER Cancer Statistics Review, 1975-2010, National Cancer Institute). The most-commonly discovered human cancers include those that arise from organs and solid tissues, e.g., colon cancer, lung cancer, breast cancer, stomach cancer, prostate cancer, and endometrial cancer. Colon cancer affects 1 in 20 people in the western hemispheres (Henderson, Nature Cell Biology, 2000, 2(9): pp. 653-60). Globally, every year 1 million new patients are diagnosed with colon cancer and half of them die from the disease (Liu et al., Cell, 2002, 108(6): pp. 837-47).

In the past decades, remarkable advancements in cancer treatment and diagnosis have been made. Treatment options for cancer includes surgery, chemotherapy, radiation therapy, and immunotherapy. Most recent immunotherapy treatment, aiming on stimulating the immune system, has particularly attracted lots of investigations. Although immunotherapy could be highly efficacious, only a proportion of patients are usually responsive to therapy, regardless of the organ of origin of the tumor. New findings in this field are clearly needed for improving immunotherapy efficacy and specificity.

Wnt-signaling controls a wide variety of cell processes, including cell fate determination, differentiation, polarity, proliferation and migration. The Wnt family of secreted proteins bind to several classes of receptors, such as the low-density lipoprotein receptor related (LRP) proteins 5 and 6 (LRP5/6), resulting in activation of several different intracellular signaling cascades, including the Wnt/beta-catenin, Wnt/calcium and Wnt/Jnk pathways. Binding of Wnt to LRP5/6 specifically activates the Wnt/beta-catenin pathway by blocking the function of a multiprotein complex that primes degradation of beta-catenin, resulting in accumulation of beta-catenin in the cytoplasm and nucleus. Nuclear beta-catenin complexes with members of the Lef/TCF family of transcription factors and activates gene expression.

Pathological states that may arise from altered stem cell function, such as degenerative diseases and cancer, are frequently associated with changes in Wnt/beta-catenin pathway activity. Indeed, hyperactivation of the Wnt/beta-catenin pathway is thought to induce premature senescence of stem cells and age-related loss of stem cell function (Brack et al., Science, 2007, Vol. 317, no. 5839, pp. 807-810; Liu et al., Science, 2007, Vol. 317, no. 5839, pp. 803-806). In cancer, hyperactivation of the Wnt/beta-catenin pathway, often in conjunction with mutations in other cell growth regulatory genes, can lead to aberrant cell growth (Reya and Clevers, Nature, 2005, 434 (7035): 843-50). Thus, many ongoing investigations are focusing on Wnt/beta-catenin pathway as a potential therapeutic target in cancer (Breuhahn et al., Oncogene, 2006, 25: 3787-3800; Greten et al., Br J Cancer, 2009, 100: 19-23). Particularly, several studies including cancer genomic sequencing projects revealed that more than 80% of colon cancers harbor a mutation in or even loss of the adenomatosis polyposis coli (APC) gene, a major suppressor of the Wnt/beta-catenin pathway (Kinzler and Vogelstein, Cell., Oct. 18, 1996; 87 (2): 159-70. Review; Sjoblom et al., Science, Oct. 13, 2006; 314 (5797): 268-74; Mann et al., Proc Natl Acad Sci USA, 1999. 96(4): pp. 1603-8). APC and proteins such as GSK3P and Axin form a complex which marks degradation of beta-catenin. Mutations in APC disrupt this complex and lead to increased levels of cytoplasmic beta-catenin and its nuclear translocation. Since beta-catenin is the most important adaptor of the Wnt signaling, it promotes expression of oncogenic factors in response to Wnt ligands.

Wnt signaling is also regulated by a number of secreted polypeptide antagonists. These antagonists include four secreted Dickkopf (DKK) proteins (Monaghan et al., Mech Dev, 1999. 87: 45-56; Krupnik et al., Gene, 1999. 238: 301-13). Among these four DKK proteins, DKK1, DKK2 and DKK4 have been demonstrated to be effective antagonists of canonical Wnt signaling (Mao et al., Nature, 2001. 411: 321-5; Semenov et al., Curr Biol, 2001. 11: 951-61; Bafico et al., Nat Cell Biol, 2001. 3: 683-6; Niehrs, Nature, 2006. 25: 7469-81) by directly binding to Wnt coreceptor LRP 5/6 with high affinities (Mao et al., Nature, 2001. 411: 321-5; Semenov et al., Curr Biol, 2001. 11: 951-61; Bafico et al., Nat Cell Biol, 2001. 3: 683-6). Although DKK1 is reported to play a crucial role in head and heart formation in vertebrate development (Niida et al., Oncogene, 2004, Nov. 4; 23 (52): 8520-6), DKK2 does not appear to play critical roles in vertebrate development. Mice lacking DKK2 have lower blood glucose (Li et al., Proc Natl Acad Sci USA, 2012. 109: 11402-7), reduced bone mass (Li et al., Nat Genet, 2005. 37: 945-52) and defective ocular surface epithelia (Gage et al., Dev Biol, 2008. 317: 310-24; Mukhopadhyay et al., Development, 2006. 133: 2149-54). Given that DKK proteins are Wnt antagonists, the conventional wisdom is that inactivation of DKK would increase Wnt activity and hence accelerate cancer formation. However, their roles in cancer formation have not been directly investigated.

The DKK molecules contain two conserved cysteine-rich domains (Niehrs, Nature, 2006. 25: 7469-81). Previously, it was shown that the second Cys-rich domains of DKK1 and DKK2 played a more important role in the inhibition of canonical Wnt signaling (Li et al., J Biol Chem, 2002. 277: 5977-81; Brott and Sokol Mol. Cell. Biol., 2002. 22: 6100-10). More recently, the structure of the second Cys-rich domain of DKK2 was solved and delineated amino acid residues on the domain that are required for DKK interactions with LRPS/6 and Kremen (Chen et al., J Biol Chem, 2008. 283: 23364-70; Wang et al., J Biol Chem, 2008. 283: 23371-5). DKK interaction with LRPS/6 underlie the primary mechanism for DKK-mediated inhibition of Wnt. Although DKK interaction with Kremen, also a transmembrane protein, was shown to facilitate DKK antagonism of Wnt signaling, this interaction may have other unresolved functions. Ala scan mutagenesis identified amino acid residues on the third YWTD repeat domain of LRPS as being important for binding to DKK1 and DKK2 (Zhang et al., Mol. Cell. Biol., 2004. 24: 4677-84). These results have been confirmed by the structural studies of a DKK1/LRP6 third and fourth YWTD repeat domain complex (Cheng et al., Nat Struct Mol Biol, 2011. 18: 1204-10; Chen et al., Dev Cell, 2011. 21: 848-61; Ahn et al., Dev Cell, 2011. 21: 862-73; Bourhis et al., Structure, 2011. 19: 1433-42). One of the structural studies also revealed a second DKK-LRP interaction site between the N-terminus of DKK and the first YWTD repeat domain of LRP (Bourhis et al., Structure, 2011. 19: 1433-42).

WO 2016/004055 discloses the discovery that inhibition of DKK2 can increase CD8+ cytotoxic T lymphocyte (CTL) activity, attenuates tumor angiogenesis, and hence suppresses tumor formation; and provides a method for treating cancer by administering to a patient an effective amount of a DKK2 gene depleting agent. WO 2017/074774 provides a method for treating cancer by administering to a patient an effective amount of a humanized anti-DKK2 antibody.

The inventors of the present disclosure have recognized that such anti-DKK2 antibodies have unfavorable biophysical properties, including low stability and aggregation tendency, which are not ideal for future clinical-stage CMC development and commercialization.

### SUMMARY OF THE INVENTION

The present disclosure relates to an anti-DKK2 antibody with improved developability and manufacturability, a composition comprising the anti-DKK2 antibody, the use of the anti-DKK2 antibody or the composition, and a method for treating diseases using the anti-DKK2 antibody or the composition.

In particular, the present disclosure provides an antibody specifically binding to a human DKK2 protein, wherein the antibody comprises a heavy chain variable region comprising complementarity-determining regions CDRH1, CDRH2, and CDRH3, and a light chain variable region comprising complementarity-determining regions CDRL1, CDRL2, and CDRL3, wherein:
(a) CDRH1 has an amino acid sequence as shown in SEQ ID NO: 1;
(b) CDRH2 has an amino acid sequence as shown in SEQ ID NO: 2 or SEQ ID NO: 3;
(c) CDRH3 has an amino acid sequence as shown in SEQ ID NO: 4;
(d) CDRL1 has an amino acid sequence as shown in SEQ ID NO: 5;
(e) CDRL2 has an amino acid sequence as shown in SEQ ID NO: 6; and
(f) CDRL3 has an amino acid sequence as shown in SEQ ID NO: 7.

The present disclosure further provides a pharmaceutical composition, comprising the antibody of the present disclosure and a pharmaceutically acceptable carrier.

The present disclosure further provides a DNA molecule encoding the heavy chain and/or light chain of the antibody of the present disclosure.

The present disclosure further provides a method for treating cancer in a subject in need thereof or for stimulating or enhancing an immune response in a subject in need thereof, the method comprising administering to the subject an effective amount of the antibody or the pharmaceutical composition of the present disclosure.

The present disclosure further provides the use of the antibody or the pharmaceutical composition of the present disclosure in the manufacture of a drug for treating cancer in a subject in need thereof or for stimulating or enhancing an immune response in a subject in need thereof.

### BRIEF DESCRIPTION OF THE DRAWINGS

The following provides a brief description of the accompanying drawings, which are used to describe the exemplary embodiments disclosed herein, rather than to limit these embodiments.

FIG. 1 shows the binding affinity of a candidate antibody (M-751, M-755 or M-763) to a human DKK2 protein compared with the parental antibody (M-747).

FIG. 2 shows tumor volumes measured on day 10, day 13 and day 16 after injection in groups treated with a candidate antibody (M-751, M-755 or M-763), a parental antibody (M-747) or a control (human polyclonal IgG or 5F8).

FIG. 3 shows mean endpoint tumor weights on day 17 after injection in groups treated with a candidate antibody (M-751, M-755 or M-763), a parental antibody (M-747) or a control (human polyclonal IgG or 5F8).

FIG. 4 shows a comparison of levels of granzyme B (an activation marker for tumor-infiltrating CD8⁺ T cells and NK1.1⁺ T cells) in mice treated with a candidate antibody (M-751, M-755, or M-763), a parental antibody (M-747), or a control (human polyclonal IgG or 5F8).

FIG. 5 shows a comparison of geometric average values of interferon γ (IFNγ, which is an activation marker for tumor-infiltrating CD8⁺ T cells and NK1.1⁺ T cells) in mice treated with a candidate antibody (M-751, M-755, or M-763), a parental antibody (M-747), or a control (human polyclonal IgG or 5F8).

FIG. 6 shows a comparison of geometric average values of CD69 (an activation marker for tumor-infiltrating CD8⁺ T cells and NK1.1⁺ T cells) in mice treated with a candidate antibody (M-751, M-755, or M-763), a parental antibody (M-747), or a control (human polyclonal IgG or 5F8).

FIG. 7 shows the SDS-PAGE analysis of the candidate antibodies (M-751 and M-755) and the parental antibody (M-747) after elution or after elution and then pH adjustment to 5.5.

### DETAILED DESCRIPTION OF EMBODIMENTS

Unless defined otherwise, all technical and scientific terms used herein have the same meanings as commonly understood by one of ordinary skill in the field to which the present disclosure belongs. Although any methods and materials similar or equivalent to those described herein can be used in the practice for testing of the present disclosure, the preferred materials and methods are described herein. In describing and claiming the present disclosure, the following terms will be used.

It should also be understood that the term used herein is for the purpose of describing particular embodiments only, and is not intended to be limiting.

The singular forms “a”, “an”, and “the” include plural forms unless the context clearly dictates otherwise.

As used in the specification and in the claims, the open-ended transitional phrases “comprise(s)”, “include(s)”, “having”, “contain(s)”, and variants thereof require the presence of the specified ingredients/steps and permit the presence of other ingredients/steps. These phrases should also be construed as disclosing the closed-ended phrases “consist of” or “consist essentially of” that permit only the specified ingredients/steps and unavoidable impurities, and exclude other ingredients/steps.

Numerical values in the specification and claims of the present application should be understood to include numerical values which are the same when reduced to the same number of significant figures, and numerical values which differ from the stated value by less than the experimental error which would occur in conventional measurement techniques of the present application.

All ranges disclosed herein are inclusive of the recited endpoint and independently combinable. Throughout the present disclosure, various aspects of the present disclosure can be presented in a range format. It should be understood that the description in range format is merely for convenience and brevity and should not be construed as an inflexible limitation on the scope of the present disclosure. Therefore, the description of the range should be considered as having explicitly disclosed all the possible subranges as well as individual numerical values within that range. For example, the description of a range such as from 1 to 6 should be considered as having explicitly disclosed subranges, such as from 1 to 3, from 1 to 4, from 1 to 5, from 2 to 4, from 2 to 6, and from 3 to 6, as well as individual values within the range, such as 1, 2, 2.7, 3, 4, 5, 5.3, and 6. All the above apply regardless of the breadth of the range.

The term “about” can be used to include any value that is changeable without changing the basic function of the value. When “about” is used in conjunction with a range, it also discloses the range defined by the absolute value of the two endpoints. For example, “from about 2 to about 4” also discloses the range of “from 2 to 4”. The term “about” may also refer to plus or minus 10% of a specified value.

The term “identity” refers to the degree of similarity between a pair of sequences (nucleotides or amino acids). The identity is determined by dividing the number of the same residues by the total number of the residues and multiplying the quotient by 100 to obtain a percentage. Gaps are excluded when assessing identity. Therefore, two copies of completely identical sequences have 100% identity, but sequences with deletion, addition or replacement may have a lower degree of identity. A person skilled in the art will recognize that there are several computer programs that can be used to determine the identity of sequences, such as those programs using algorithms such as BLAST. BLAST nucleotide search is performed using the NBLAST program, and BLAST protein search is performed using the BLASTP program, and default parameters of each program are used.

Two different sequences can be different from each other without affecting the overall function of the protein encoded by the sequence. In this regard, it is well known in the art that chemically similar amino acids can be substituted with each other, usually without changing their functions. Relevant properties can include acidity/alkalinity, polarity/non-polarity, electric charge, hydrophobicity, and chemical structure. For example, alkaline residues Lys and Arg are considered chemically similar and often replace each other. Other examples are, for example, acidic residues Asp and Glu, hydroxyl residues Ser and Thr, aromatic residues Tyr, Phe, and Trp, and non-polar residues Ala, Val, Ile, Leu, and Met. These replacements are considered “conservative”. Similarly, nucleotide codons and acceptable changes are also known in the art. For example, codons ACT, ACC, ACA, and ACG all encode an amino acid threonine, that is, the third nucleotide can be changed without changing the obtained amino acid. The similarity is determined by dividing the number of the similar residues by the total number of the residues and multiplying the quotient by 100 to obtain a percentage. It should be noted that the measurement of the similarity and the identity are indicative of different properties.

As used herein, the terms “control”, or “reference” are used interchangeably, and refer to a value that is used as a standard of comparison (e.g., expression level of DKK2 in a healthy subject).

A “subject” or “patient”, as used herein, may be a human or non-human mammal. Non-human mammals include, for example, livestock and pets, such as ovine, bovine, porcine, canine, feline and murine mammals. Preferably, the subject is human.

As used herein, the term “compound” includes a macromolecular compound (e.g., an antibody) and a small molecular compound.

As used herein, the term “activation” refers to the state of a cell in which distinct biochemical or morphological changes are induced following sufficient cell surface moiety ligation. In the context of T cells, such activation refers to the state of a T cell that has been sufficiently stimulated to induce cellular proliferation. Activation of a T cell may also induce cytokine production and effect regulatory or cytolytic effector functions. Within the context of other cells, this term infers either up or down regulation of a particular physico-chemical process. The term “activated T cell” means a T cell that is currently undergoing cell division, cytokine production, effecting regulatory or cytolytic effector functions, and/or has recently undergone the process of “activation”.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids of which a protein or peptide sequence is comprised. Polypeptides include any peptide or protein comprising two or more amino acids joined to each other by peptide bonds. As used herein, the term refers to both short chains, which are also commonly referred to in the art as peptides, oligopeptides and oligomers, for example, and to longer chains, which are generally referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, and fusion proteins. The polypeptides include natural peptides, recombinant peptides, synthetic peptides, or a combination thereof.

The term “immunotherapeutic agent” as used herein is meant to include any agent that modulates the immune system of a patient. “Immunotherapy” refers to the treatment that alters the immune system of a patient.

The term “treatment” in the context of the present disclosure is meant to comprise therapeutic treatment, as well as prophylactic or suppressive measures for a disease or condition. Thus, for example, the term treatment comprises administrating an agent prior to or following the onset of a disease or condition, thereby preventing or eliminating all signs of the disease or condition. As another example, administration of the agent after clinical manifestation of the disease to combat the symptoms of the disease comprises “treatment” of the disease. This includes prevention of cancer.

“DKK protein” refers to a member of the DKK family of proteins that contains one or more cysteine-rich domains. The DKK family of proteins includes DKK1, DKK2, DKK3 and DKK4, and any other protein sufficiently related to one or more of these proteins at the sequence level, structurally or functionally. This family of proteins is described, e.g., in Krupnik et al. (1999) Gene 238:301. Allelic variants and mutants of DKK proteins such as those recited herein are also included in this definition.

“Humanized” forms of non-human (e.g., murine) antibodies refer to chimeric immunoglobulins, or immunoglobulin chains or fragments thereof (e.g., Fv, Fab, Fab′, F(ab′)2 or other antigen-binding subsequences of the antibodies) which contain minimal sequences derived from non-human immunoglobulins. In most cases, humanized antibodies are human immunoglobulins (recipient antibodies) in which residues from a complementarity-determining region (CDR) of a recipient are replaced by residues from a CDR of the non-human species such as mice, rats, or rabbits (donor antibodies) with the desired specificity, affinity, and capacity. In some cases, Fv framework region (FR) residues of the human immunoglobulin may be replaced by corresponding non-human residues. Furthermore, humanized antibodies can comprise residues that are not found in either the recipient antibody or in the imported CDR or framework sequences. These modifications are made to further improve and optimize antibody performance. In general, the humanized antibody will comprise substantially all of at least one, and typically two, variable domains, in which all or substantially all of the CDR regions correspond to those of the non-human immunoglobulins and all or substantially all of the FR regions are those of a human immunoglobulin sequence. The humanized antibody optimally also will comprise at least a portion of an immunoglobulin constant region (Fc), typically that of a human immunoglobulin Fc. For further details, see Jones et al., Nature, 321: 522-525, 1986; Reichmann et al., Nature, 332: 323-329, 1988; and Presta, Curr. Op. Struct. Biol., 2: 593-596, 1992.

The term “cancer” as used herein, includes any malignant tumor including, but not limited to, carcinoma and sarcoma. Cancer arises from the uncontrolled and/or abnormal division of cells and then invades and destroys the surrounding tissues. As used herein, “proliferation” refers to cells undergoing mitosis. As used herein, “metastasis” refers to the distant spread of a malignant tumor from its origin. Cancer cells may metastasize through the bloodstream, through the lymphatic system, across body cavities, or any combination thereof.

As used herein, the term “pharmaceutical composition” refers to a mixture of at least one of the compounds which are useful in the present disclosure and other chemical components, such as a carrier, a stabilizer, a diluent, a dispersant, a suspension, a thickener, and/or an excipient. The pharmaceutical composition facilitates the administration of the compound to an organism. There are many techniques for administering compounds in the art, including but not limited to: intravenous, oral, aerosol, parenteral, eye, lung and topical administration.

The term “pharmaceutically acceptable carrier” includes a pharmaceutically acceptable salt, and a pharmaceutically acceptable material, composition or carrier, such as a liquid or solid filler, diluent, excipient, solvent or encapsulating material, and involves carrying or transporting one or more compounds of the present disclosure within a subject or carrying or transporting the one or more compounds of the present disclosure to a subject so as to perform its intended functions. Typically, such compounds are carried or transported from one organ, or portion of the body, to another organ, or portion of the body. Each salt or carrier must be “acceptable” regarding compatibility with other components of the preparation and not harmful to the subject. Some examples of materials that can be used as pharmaceutically acceptable carriers include: sugar, such as lactose, glucose, and sucrose; starch, such as corn starch and potato starch; cellulose and derivatives thereof, such as sodium carboxymethyl cellulose, ethyl cellulose, and cellulose acetate; powdered tragacanth gum; malt; gelatin; talc; an excipient, such as cocoa butter and suppository waxes; oil, such as peanut oil, cottonseed oil, safflower oil, sesame oil, olive oil, corn oil and soybean oil; diols, such as propylene glycol; polyols, such as glycerol, sorbitol, mannitol, and polyethylene glycol; esters, such as ethyl oleate and ethyl laurate; agar; a buffering agent, such as magnesium hydroxide and aluminum hydroxide; alginic acid; pyrogen-free water; isotonic saline; a Ringer's solution; ethanol; a phosphate buffer solution; a diluent; a granulating agent; a lubricant; a binder; a disintegrant; a wetting agent; an emulsifier; a colorant; a mold release agent; a coating agent; a sweetener; a flavoring agent; an aromatizing agent; a preservative; an antioxidant; a plasticizer; a gelatinizer; a thickener; a hardener; a setting agent; a suspension; a surfactant; a humectant; a carrier; a stabilizer; and other non-toxic compatible substances used in pharmaceutical preparations, or any combination thereof. As used herein, “pharmaceutically acceptable carrier” also includes any and all coatings, antibacterial and antifungal agents, absorption delaying agents, etc. that are compatible with the activity of the compound, and are physiologically acceptable to the subject. Supplementary active compounds may also be incorporated into the compositions.

The term “antibody” refers to a protein used to recognize target antigens by the immune system. The basic functional unit of the antibody is an immunoglobulin monomer. The monomer consists of two identical heavy chains and two identical light chains that form a Y-shaped protein. Each light chain consists of one constant domain and one variable domain. In the light chain, the constant domain can also be called a “constant region”, and the variable domain can also be called a “variable region”. Each heavy chain consists of one variable domain and three or four constant domains. In the heavy chain, the constant domains together are called “constant regions”, while the variable domain can also be called a “variable region”. The arms of Y are called fragments, antigen binding (Fab) regions, and each arm is called a Fab fragment. Each Fab fragment consists of one constant domain and one variable domain from the heavy chain, and one constant domain and one variable domain from the light chain. The base of Y is called an Fc region, which consists of two or three constant domains from each heavy chain. The variable domains of the heavy chain and the light chain in the Fab regions are parts for binding antigens of the antibody (such as DKK2 in the present disclosure). More specifically, complementarity-determining regions (CDRs) of the variable domains bind their antigens (such as DKK2). In the amino acid sequence of each variable domain, there are three discontinuous CDRs. The term “complete” used herein refers to an antibody containing Fab and Fc regions.

The “antibody heavy chain” used herein refers to the larger of two types of polypeptide chains that exist in the antibody molecule in its natural conformation and usually determines the type of the antibody.

The “antibody light chain” used herein refers to the smaller of two types of polypeptide chains that exist in the antibody molecule in its natural conformation. κ and λ light chains refer to two major isotypes of the antibody light chain.

The term “antigen” or “Ag” as used herein is defined as a molecule that elicits an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. Therefore, a skilled artisan will understand that any DNA, which comprises a nucleotide sequence or a partial nucleotide sequence encoding a protein that elicits an immune response encodes the term “antigen” as used herein. Furthermore, a person skilled in the art will understand that an antigen needs not to be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen needs not to be encoded by a “gene” at all. It is readily apparent that an antigen can be synthesized or can be derived from a biological sample. Such a biological sample can include, but is not limited to a tissue sample, a tumor sample, a cell or a biological fluid.

The immune system is balanced between activation and suppression. Evasion of immunological surveillance is one of the prerequisites for tumor formation. One of the ways for tumors to evade immunological surveillance is to produce elevated amount of immunosuppressive molecules. Increasing number of immunosuppressive molecules and mechanisms have been identified over the years. Neutralization of these immunosuppressive molecules has been shown to be efficacious in treating various malignancies.

The present disclosure relates to the discovery of DKK2 that suppresses natural killer (NK) cell and CD8⁺ cytotoxic T lymphocyte (CTL) activities. DKK2 is a secreted protein, which can inhibit beta-catenin-mediated Wnt signaling, alter non-beta-catenin-mediated Wnt activity, and may also have Wnt-independent functions. DKK2 is expressed in many tissues and is upregulated in human colorectal, gastric intestinal, liver, kidney, and pancreatic cancers. Experimental evidence described below indicates that DKK2 antibodies are key immunomodulators for treating cancers in which DKK2 is expressed. Thus, DKK2 is a promising target for treating these cancers.

In some embodiments, an anti-DKK2 antibody can be humanized, wherein specific sequences or regions of the antibody are modified to increase similarity to an antibody naturally produced in a human. For example, in the present disclosure, the antibody or fragment thereof may comprise a non-human mammalian scFv. In one embodiment, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. 592,106 and EP 519,596; Padlan, 1991, Molecular Immunology, 28(4/5): 489-498; Studnicka et al., 1994, Protein Engineering, 7(6):805-814; and oguska et al., 1994, PNAS, 91:969-973, each of which is incorporated herein in its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213 and 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein by reference in its entirety. Often, framework residues in the framework regions will be substituted with the corresponding residue from the CDR donor antibody to alter, preferably improve, antigen binding. These framework substitutions are identified by methods well-known in the art, e.g., by modeling of the interactions between the CDR and framework residues to identify framework residues important for antigen binding and by sequence comparison to identify unusual framework residues at particular positions. (See, e.g., Queen et al., U.S. Pat. No. 5,585,089; and Riechmann et al., 1988, Nature, 332:323, which are incorporated herein by reference in their entirety).

A humanized antibody has one or more amino acid residues which are introduced into the antibody from a non-human source. These non-human amino acid residues are often referred to as “import” residues, which are typically taken from an “import” variable domain. Thus, humanized antibodies comprise one or more CDRs from non-human immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences with the corresponding sequences of a human antibody, i.e., CDR-grafting (EP 239,400; PCT Publication No. WO 91/09967; and U.S. Pat. Nos. 4,816,567, 6,331,415, 5,225,539, 5,530,101, 5,585,089, and 6,548,640, the contents of which are incorporated herein by reference in their entirety). In such humanized chimeric antibodies, substantially less than an intact human variable domain has been substituted by the corresponding sequence from a non-human species. In practice, humanized antibodies are typically human antibodies in which some CDR residues and some possible framework (FR) residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy chain variable domains, which are used for preparing the humanized antibodies is made to reduce antigenicity. According to the so-called “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference in their entirety). Another method uses a particular framework derived from the consensus sequence of all human antibodies with a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference in their entirety).

Antibodies can be humanized with retention of high affinity for the target antigen and other favorable biological properties. According to one aspect of the present disclosure, humanized antibodies are prepared by a method comprising analyzing the parental sequences and various conceptual humanized products using three-dimensional models of the parental and humanized sequences. Three-dimensional immunoglobulin models are commonly available and are familiar to a person skilled in the art. Computer programs which illustrate and display probable three-dimensional conformational structures of selected candidate immunoglobulin sequences are available. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity to the original antibody. However, when using certain methods of humanization, the affinity and/or specificity regarding the binding of the antibody to the target antigen may be increased by using the method of “directed evolution”, as described by Wu et al., J. Mol. Biol., 294:151 (1999), the contents of which are incorporated herein by reference in their entirety.

The antibody of the present disclosure can be assessed for immunospecific binding by any method known in the art. The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Current Protocols in Molecular Biology, (Ausubel et al., ed.), Greene Publishing Associates and Wiley-Interscience, New York, 2002).

In some embodiments, the present disclosure relates to an antibody specifically binding to a human DKK2 protein, wherein the antibody comprises a heavy chain variable region comprising complementarity-determining regions CDRH1, CDRH2, and CDRH3, and a light chain variable region comprising complementarity-determining regions CDRL1, CDRL2, and CDRL3, wherein:
(a) CDRH1 has an amino acid sequence as shown in SEQ ID NO: 1;
(b) CDRH2 has an amino acid sequence as shown in SEQ ID NO: 2 or SEQ ID NO: 3;
(c) CDRH3 has an amino acid sequence as shown in SEQ ID NO: 4;
(d) CDRL1 has an amino acid sequence as shown in SEQ ID NO: 5;
(e) CDRL2 has an amino acid sequence as shown in SEQ ID NO: 6; and
(f) CDRL3 has an amino acid sequence as shown in SEQ ID NO: 7. In some embodiments, the CDRH1, CDRH2, CDRH3, CDRL1, CDRL2 and CDRL3 of the antibody have amino acid sequences as shown in SEQ ID NO: 1, SEQ ID NO: 2, SEQ ID NO: 4, SEQ ID NO: 5, SEQ ID NO: 6 and SEQ ID NO: 7, respectively.

In some embodiments, the CDRH1, CDRH2, CDRH3, CDRL1, CDRL2 and CDRL3 of the antibody have amino acid sequences as shown in SEQ ID NO: 1, SEQ ID NO: 3, SEQ ID NO: 4, SEQ ID NO: 5, SEQ ID NO: 6 and SEQ ID NO: 7, respectively.

In some embodiments, the light chain variable region of the antibody comprises an amino acid sequence as shown in SEQ ID NO: 8 or SEQ ID NO: 12, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 8 or SEQ ID NO: 12. In some embodiments, the light chain variable region of the antibody comprises an amino acid sequence as shown in SEQ ID NO: 8. In some embodiments, the light chain variable region of the antibody comprises an amino acid sequence as shown in SEQ ID NO: 12. In some embodiments, the light chain variable region of the antibody has an amino acid sequence as shown in SEQ ID NO: 8. In some embodiments, the light chain variable region of the antibody has an amino acid sequence as shown in SEQ ID NO: 12.

In some embodiments, the heavy chain variable region of the antibody comprises an amino acid sequence as shown in SEQ ID NO: 10 or SEQ ID NO: 14, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 10 or SEQ ID NO: 14. In some embodiments, the heavy chain variable region of the antibody comprises an amino acid sequence as shown in SEQ ID NO: 10. In some embodiments, the heavy chain variable region of the antibody comprises an amino acid sequence as shown in SEQ ID NO: 14. In some embodiments, the heavy chain variable region of the antibody has an amino acid sequence as shown in SEQ ID NO: 10. In some embodiments, the heavy chain variable region of the antibody has an amino acid sequence as shown in SEQ ID NO: 14.

In some embodiments, the light chain variable region of the antibody comprises an amino acid sequence as shown in SEQ ID NO: 8, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 8, and the heavy chain variable region of the antibody comprises an amino acid sequence as shown in SEQ ID NO: 10, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 10. In some embodiments, the light chain variable region of the antibody comprises an amino acid sequence as shown in SEQ ID NO: 12, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 12, and the heavy chain variable region of the antibody comprises an amino acid sequence as shown in SEQ ID NO: 14, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 14. In some embodiments, the light chain variable region of the antibody comprises an amino acid sequence as shown in SEQ ID NO: 8 and the heavy chain variable region of the antibody comprises an amino acid sequence as shown in SEQ ID NO: 10. In some embodiments, the light chain variable region of the antibody comprises an amino acid sequence as shown in SEQ ID NO: 12 and the heavy chain variable region of the antibody comprises an amino acid sequence as shown in SEQ ID NO: 14. In some embodiments, the light chain variable region of the antibody has an amino acid sequence as shown in SEQ ID NO: 8 and the heavy chain variable region of the antibody has an amino acid sequence as shown in SEQ ID NO: 10. In some embodiments, the light chain variable region of the antibody has an amino acid sequence as shown in SEQ ID NO: 12 and the heavy chain variable region of the antibody has an amino acid sequence as shown in SEQ ID NO: 14.

In some embodiments, the light chain variable region of the antibody and/or the heavy chain variable region of the antibody are a part of a single-chain variable fragment (scFv), an F(ab′)2 fragment, an Fab or Fab′ fragment, a bivalent antibody, a trivalent antibody, a tetravalent antibody or a monoclonal antibody.

The scFv includes a light chain variable region and a heavy chain variable region that are usually joined together by a linker. The linker is usually about 10 amino acids to about 25 amino acids in length (although it does not have to be in this range). An N-terminal of one variable domain is connected to a C-terminal of other variable domains. It is desirable that the scFv is subjected to PEGylation (i.e., treated with polyethylene glycol) to increase its size, as certolizumab pegol. Two scFvs can be bonded together by another linker to produce a tandem scFv.

If the light chain variable region and the heavy chain variable region are joined together by a short linker to form scFv, the two variable domains cannot be overlapped, otherwise the scFv will undergo dimerization to form a bivalent antibody. Even shorter linkers can lead to the formation of trimers (i.e., a trivalent antibody) and tetramers (i.e., a tetravalent antibody).

A complete monoclonal antibody consists of two heavy chains and two light chains. Besides, each light chain and each heavy chain contain variable domains. Each light chain is joined with a heavy chain. Two heavy chains are joined together in a hinge region. If a heavy chain constant region below the hinge region is removed, an F(ab′)2 fragment containing a total of four variable domains is generated. The F(ab′)2 fragment can be divided into two Fab′ fragments. The Fab′ fragments contain sulfhydryl groups from the hinge region. When the heavy chain constant region above the hinge region is removed, an Fab fragment is formed and does not contain sulfhydryl groups from the hinge region. However, all these fragments contain light chain variable regions and heavy chain variable regions.

In some embodiments, the antibody of the present disclosure is a complete monoclonal antibody formed by combining the above-mentioned light chains and heavy chains containing variable regions/domains with human constant regions. The heavy chain constant region can be any human isotypes, including IgA1, IgA2, IgD, IgE, IgG1, IgG2, IgG3, IgG4 or IgM, preferably IgG4. The human light chain constant region can be κ or λ isotypes, preferably a κ isotype. In some embodiments, the heavy chain constant region is an IgG4 isotype and the light chain constant region is a κ isotype.

In some embodiments, the light chain of the antibody comprises an amino acid sequence as shown in SEQ ID NO: 9 or SEQ ID NO: 13, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 9 or SEQ ID NO: 13.

In some embodiments, the light chain of the antibody comprises an amino acid sequence as shown in SEQ ID NO: 9. In some embodiments, the light chain of the antibody comprises an amino acid sequence as shown in SEQ ID NO: 13. In some embodiments, the light chain of the antibody has an amino acid sequence as shown in SEQ ID NO: 9. In some embodiments, the light chain of the antibody has an amino acid sequence as shown in SEQ ID NO: 13.

In some embodiments, the heavy chain of the antibody comprises an amino acid sequence as shown in SEQ ID NO: 11 or SEQ ID NO: 15, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 11 or SEQ ID NO: 15. In some embodiments, the heavy chain of the antibody comprises an amino acid sequence as shown in SEQ ID NO: 11. In some embodiments, the heavy chain of the antibody comprises an amino acid sequence as shown in SEQ ID NO: 15. In some embodiments, the heavy chain of the antibody has an amino acid sequence as shown in SEQ ID NO: 11. In some embodiments, the heavy chain of the antibody has an amino acid sequence as shown in SEQ ID NO: 15.

In some embodiments, the light chain of the antibody comprises an amino acid sequence as shown in SEQ ID NO: 9, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 9, and the heavy chain of the antibody comprises an amino acid sequence as shown in SEQ ID NO: 11, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 11. In some embodiments, the light chain of the antibody comprises an amino acid sequence as shown in SEQ ID NO: 13, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 13, and the heavy chain of the antibody comprises an amino acid sequence as shown in SEQ ID NO: 15, or an amino acid sequence having at least 80%, at least 85%, at least 90%, at least 95%, at least 96%, at least 97%, at least 98%, at least 99%, or at least 99.5% sequence identity to the amino acid sequence as shown in SEQ ID NO: 15. In some embodiments, the light chain of the antibody comprises an amino acid sequence as shown in SEQ ID NO: 9 and the heavy chain of the antibody comprises an amino acid sequence as shown in SEQ ID NO: 11. In some embodiments, the light chain of the antibody comprises an amino acid sequence as shown in SEQ ID NO: 13 and the heavy chain of the antibody comprises an amino acid sequence as shown in SEQ ID NO: 15. In some embodiments, the light chain of the antibody has an amino acid sequence as shown in SEQ ID NO: 9 and the heavy chain of the antibody has an amino acid sequence as shown in SEQ ID NO: 11. In some embodiments, the light chain of the antibody has an amino acid sequence as shown in SEQ ID NO: 13 and the heavy chain of the antibody has an amino acid sequence as shown in SEQ ID NO: 15.

In one aspect, the present disclosure contemplates that the antibody of the present disclosure may be used in combination with a therapeutic agent such as an anti-tumor agent, including but not limited to a chemotherapeutic agent, an immunotherapeutic agent, an anti-cell proliferation agent or any combination thereof. For example, any conventional chemotherapeutic agents of the following non-limiting exemplary classes are included in the present disclosure: alkylating agents; nitrosoureas; antimetabolites; antitumor antibiotics; plant alkaloids; taxanes; hormonal agents; and miscellaneous agents.

Alkylating agents are so named because of their ability to add alkyl groups to many electronegative groups under conditions present in cells, thereby interfering with DNA replication so as to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, mesalazine hydrochloride, procarbazine, thiotepa, and uracil mustard.

Antimetabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include aclacinomycin, actinomycin, anthramycin, azaserine, bleomycins, cactinomycin, calicheamicin, carubicin, caminomycin, carzinophilin, chromomycin, dactinomycin, daunorubicin, detorubicin, 6-diazo-5-oxo-L-norleucine, doxorubicin, epirubicin, esorubicin, idarubicin, marcellomycin, mitomycins, mitoxantrone, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin.

Plant alkaloids inhibit or stop mitosis or inhibit enzymes that prevent cells from making proteins needed for cell growth. Common plant alkaloids include vinblastine, vincristine, vindesine, and vinorelbine. However, the present disclosure should not be construed as being limited solely to these plant alkaloids.

Taxanes affect cell structures called microtubules that are important in cellular functions. In normal cell growth, microtubules are formed when a cell starts dividing, but once the cell stops dividing, the microtubules are broken down or destroyed. Taxanes prohibit the microtubules from breaking down such that the cancer cells become so clogged with microtubules that they cannot grow or divide. Non-limiting exemplary taxanes include paclitaxel and docetaxel.

Hormonal agents and hormone-like drugs are utilized for certain types of cancer, including, for example, leukemia, lymphoma, and multiple myeloma. They are often used with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to a therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutic agents such as bleomycin, hydroxyurea, L-asparaginase, and procarbazine.

Other examples of chemotherapeutic agents include, but are not limited to, the following and their pharmaceutically acceptable salts, acids and derivatives: nitrogen mustards such as chlorambucil, clozapine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defofamine; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK@ razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside (“Ara-C”); cyclophosphamide; thiotepa; taxanes, e.g. paclitaxel (TAXOLO, Bristol-Myers Squibb Oncology, Princeton, N.J.) and docetaxel (TAXOTERE, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriamine pentaacetic acid, irofulven, diphtheria toxin, mitogillin, ricin, botulinum toxin, cholera toxin, saporin 6, or a combination thereof.

An immunotherapeutic agent may be, but is not limited to, an interleukin-2 or other cytokines, an inhibitor of programmed cell death protein 1 (PD-1) signaling such as a monoclonal antibody that binds to PD-1, Ipilimumab. The immunotherapeutic agent can also block cytotoxic T lymphocytes associated antigen A-4 (CTLA-4) signaling and it can also relate to cancer vaccines and dendritic cell-based therapies.

The immunotherapeutic agent can further be NK cells that are activated and expanded by means of cytokine treatment or by means of transferring exogenous cells by adoptive cell therapy and/or by hematopoietic stem cell transplantation. NK cells suitable for adoptive cell therapy can be derived from different sources, including ex vivo expansion of autologous NK cells, unstimulated or expanded allogeneic NK cells from peripheral blood, CD34+ hematopoietic progenitors from peripheral blood and umbilical cord blood, and NK-cell lines. Genetically modified NK cells expressing chimeric antigen receptors or cytokines are also contemplated in the present disclosure. Another immunotherapeutic agent useful for the present disclosure is an agent based on adoptive T cell therapy (ACT), wherein tumor-infiltrating lymphocytes (TILs) are administered to patients. The administered T cells can be genetically engineered to express tumor-specific antigen receptors such as chimeric antigen receptors (CARs), which recognize cell-surface antigens in a non-major histocompatibility (MHC)-restricted manner; or they can be traditional alpha beta TCRs, which recognize epitopes of intracellular antigens presented by MHC molecules.

In some embodiments, the present disclosure relates to a DNA molecule encoding the heavy chain and/or light chain of the antibody of the present disclosure. In some embodiments, the present disclosure relates to a DNA molecule encoding an amino acid sequence as shown in SEQ ID NO: 9. In some embodiments, the present disclosure relates to a DNA molecule encoding an amino acid sequence as shown in SEQ ID NO: 11. In some embodiments, the present disclosure relates to a DNA molecule encoding an amino acid sequence as shown in SEQ ID NO: 13. In some embodiments, the present disclosure relates to a DNA molecule encoding an amino acid sequence as shown in SEQ ID NO: 15. In some embodiments, the present disclosure relates to a DNA molecule encoding an amino acid sequence as shown in SEQ ID NO: 9 and an amino acid sequence as shown in SEQ ID NO: 11. In some embodiments, the present disclosure relates to a DNA molecule encoding an amino acid sequence as shown in SEQ ID NO: 13 and an amino acid sequence as shown in SEQ ID NO: 15.

In some embodiments, the present disclosure relates to a pharmaceutical composition, comprising the antibody of the present disclosure and a pharmaceutically acceptable carrier.

In some embodiments, the pharmaceutical composition comprises a pharmaceutically acceptable carrier. The carrier acts as a medium for delivering an antibody. Examples of the pharmaceutically acceptable carrier include a liquid carrier (such as water, oil, and alcohols) in which an antibody can be dissolved or suspended.

The pharmaceutical composition may also include an excipient. Specifically, the excipient includes a buffering agent, a surfactant, a preservative, a filler, a polymer, and a stabilizer, which can be used together with the antibody. The buffering agent is used to control the pH value of the composition. The surfactant is used to stabilize proteins, inhibit protein aggregation, inhibit protein adsorption to the surface, and assist in protein refolding. Examples of the surfactant include Tween Tween 20, Brij 35, Triton X-10, PluronicF127, and sodium dodecyl sulfate. The preservative is used to inhibit microbial growth. Examples of the preservative include benzyl alcohol, m-cresol, and phenol. The filler is used during freeze-drying to increase volume. The hydrophilic polymer (such as dextran, hydroxyethyl starch, polyethylene glycol, and gelatin) can be used to stabilize proteins. The polymer with nonpolar parts (such as polyethylene glycol polymers) can also be used as a surfactant. The protein stabilizer may include polyols, sugars, amino acids, amines, and salts. The suitable sugars include sucrose and trehalose. The amino acids include histidine, arginine, glycine, methionine, proline, lysine, glutamate, and a mixture thereof. Proteins such as human serum albumin can also be competitively adsorbed on the surface and reduce antibody aggregation. It should be noted that specific molecules can be used for many purposes. For example, histidine can be used as a buffering agent and an antioxidant. Glycine can be used as a buffering agent and a filler.

In one embodiment, the antibody or the pharmaceutical composition of the present disclosure may be administered to a subject at a dose of 0.01-50 mg/kg/day. In one embodiment, the antibody or the pharmaceutical composition of the present disclosure may be administered to a subject at a dose of 0.1-40 mg/kg/day. In one embodiment, the antibody or the pharmaceutical composition of the present disclosure may be administered to a subject at a dose of 1-30 mg/kg/day. In one embodiment, the antibody or the pharmaceutical composition of the present disclosure may be administered to a subject at a dose of 10-20 mg/kg/day.

In one embodiment, the antibody or the pharmaceutical composition of the present disclosure may be administered to a subject at an interval between 1-14 days. In one embodiment, the antibody or the pharmaceutical composition of the present disclosure may be administered to a subject at an interval between 1-10 days. In one embodiment, the antibody or the pharmaceutical composition of the present disclosure may be administered to a subject at an interval between 1-7 days. In one embodiment, the antibody or the pharmaceutical composition of the present disclosure may be administered to a subject at an interval between 1-5 days. In one embodiment, the antibody or the pharmaceutical composition of the present disclosure may be administered to a subject at an interval between 1-3 days. In one embodiment, the antibody or the pharmaceutical composition of the present disclosure may be administered to a subject at an interval between 2-14 days. In one embodiment, the antibody or the pharmaceutical composition of the present disclosure may be administered to a subject at an interval between 2-10 days. In one embodiment, the antibody or the pharmaceutical composition of the present disclosure may be administered to a subject at an interval between 2-7 days. In one embodiment, the antibody or the pharmaceutical composition of the present disclosure may be administered to a subject at an interval between 2-5 days. In one embodiment, the antibody or the pharmaceutical composition of the present disclosure may be administered to a subject at an interval between 2-3 days. In one embodiment, the antibody or the pharmaceutical composition of the present disclosure may be administered to a subject at an interval between 3-14 days. In one embodiment, the antibody or the pharmaceutical composition of the present disclosure may be administered to a subject at an interval between 3-10 days. In one embodiment, the antibody or the pharmaceutical composition of the present disclosure may be administered to a subject at an interval between 3-7 days. In one embodiment, the antibody or the pharmaceutical composition of the present disclosure may be administered to a subject at an interval between 3-5 days. In one embodiment, the antibody or the pharmaceutical composition of the present disclosure may be administered to a subject at an interval between 5-14 days. In one embodiment, the antibody or the pharmaceutical composition of the present disclosure may be administered to a subject at an interval between 5-10 days. In one embodiment, the antibody or the pharmaceutical composition of the present disclosure may be administered to a subject at an interval between 5-7 days. In one embodiment, the antibody or the pharmaceutical composition of the present disclosure may be administered to a subject at an interval between 7-14 days. In one embodiment, the antibody or the pharmaceutical composition of the present disclosure may be administered to a subject at an interval between 7-10 days.

In one embodiment, the antibody or the pharmaceutical composition of the present disclosure may be administered to a subject at a dose of 0.01-50 mg/kg/day with an interval between 1-14 days. In one embodiment, the antibody or the pharmaceutical composition of the present disclosure may be administered to a subject at a dose of 0.1-40 mg/kg/day with an interval between 1-14 days. In one embodiment, the antibody or the pharmaceutical composition of the present disclosure may be administered to a subject at a dose of 1-30 mg/kg/day with an interval between 1-14 days. In one embodiment, the antibody or the pharmaceutical composition of the present disclosure may be administered to a subject at a dose of 10-20 mg/kg/day with an interval between 1-14 days.

The relative amounts of the active ingredient, the pharmaceutically acceptable carrier, and any additional ingredients in a pharmaceutical composition of the present disclosure will vary, depending upon the identity, size, and condition of the subject treated and further depending upon the route by which the composition is to be administered. By way of example, the composition may comprise 0.1%-100% (w/w) of the active ingredient.

The antibody or the pharmaceutical composition of the present disclosure may be suitably developed for inhalational, oral, rectal, vaginal, parenteral, topical, transdermal, pulmonary, intranasal, buccal, ophthalmic, intrathecal, intravenous or another administration route. Other contemplated preparations include projected nanoparticles, liposomal articles, resealed erythrocytes containing the active ingredient, and immuno-based preparations. The administration route is readily apparent to a person skilled in the art and depends upon a number of factors including the type and severity of the disease being treated, the type and age of the veterinary or human patient being treated, and the like.

The preparations of the antibody or the pharmaceutical composition of the present disclosure may be prepared by any method known or hereafter developed in the art of pharmacology. In general, such preparation methods include the step of combining the active ingredient with a carrier or one or more other auxiliary ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or an appropriate fraction of such a dosage such as, for example, one-half or one-third of such a dosage. The unit dosage form may be used in a single daily dose or one of multiple daily doses (e.g., about 1 to 4 or more times per day). When multiple daily doses are used, the unit dosage form may be the same or different for each dose.

Although the descriptions of the antibody or the pharmaceutical composition of the present disclosure are principally directed to pharmaceutical compositions suitable for ethical administration to humans, it should be understood by a person skilled in the art that such antibody or pharmaceutical composition is generally suitable for administration to animals of all sorts. Modification of antibodies or pharmaceutical compositions suitable for administration to humans in order to render the antibodies or pharmaceutical compositions suitable for administration to animals of all sorts is known to all, and the ordinarily skilled veterinary pharmacologist can design and perform such modification with merely ordinary, if any, experimentation. Subjects to which the antibody or the pharmaceutical composition of the present disclosure is administrated is contemplated, including, but not limited to, humans and other primates, mammals including commercially relevant mammals such as cattle, pigs, horses, sheep, cats, and dogs.

In one embodiment, the pharmaceutical composition is formulated using one or more pharmaceutically acceptable excipients or carriers. In one embodiment, the pharmaceutical composition comprises a therapeutically effective amount of humanized anti-DKK2 antibody and a pharmaceutically acceptable carrier. Pharmaceutically acceptable carriers, which are available, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, and thimerosal. In many cases, it is preferable to include isotonic agents, for example, sugars, sodium chloride, or polyols such as mannitol and sorbitol, in the composition. Prolonged absorption of the injectable compositions may be achieved by including in the composition an agent which delays absorption, for example, aluminum monostearate or gelatin.

Preparations may be used in mixtures with conventional excipients, i.e., pharmaceutically acceptable organic or inorganic carrier substances suitable for oral, parenteral, nasal, intravenous, subcutaneous, or enteral administration, or any other suitable mode of administration known to the art. The pharmaceutical articles may be sterilized and if desired mixed with auxiliary agents, e.g., lubricants, preservatives, stabilizers, wetting agents, emulsifiers, salts for influencing osmotic pressure, buffering agents, colorants, flavoring agents and/or aromatic substances. They may also be combined where desired with other active agents, e.g., other analgesic agents.

The composition of the present disclosure may comprise a preservative accounting for about 0.005% to 2.0% of the total weight of the composition. The preservative is used to prevent spoilage in the case of exposure to contaminants in the environment. Examples of preservatives useful in accordance with the present disclosure included but are not limited to those selected from benzyl alcohol, sorbic acid, parabens, iminourea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% by weight of the total weight of the composition, more preferably BHT in the range of 0.03% to 0.1% by weight of the total weight of the composition. Preferably, the chelating agent is present in an amount of from to 0.5% by weight of the total weight of the composition. Particularly preferred chelating agents include edetate salts (e.g. disodium edetate) and citric acid in the range of about 0.01-0.20% and more preferably in the range of 0.02-0.10% by weight of the total weight of the composition. The chelating agent is useful for chelating metal ions in the composition which may be detrimental to the shelf life of the preparation. For some compounds, BHT and disodium edetate are the particularly preferred antioxidant and chelating agent, respectively, but as known to a person skilled in the art, other suitable and equivalent antioxidants and chelating agents may therefore be substituted.

The administration regimen may affect what constitutes an effective amount. For example, the therapeutic preparations may be administered to the patient either prior to or after a surgical intervention related to cancer, or shortly after the patient was diagnosed with cancer. Further, several divided dosages, as well as staggered dosages may be administered daily or sequentially, or the dose may be continuously infused, or may be a single bolus injection. Further, the dosages of the therapeutic preparations may be proportionally increased or decreased as indicated by the exigencies under the therapeutic or prophylactic situation.

Administration of the antibody or composition of the present disclosure to a patient, preferably a mammal, more preferably a human, may be carried out using known procedures at a dosage for a period of time effective to treat cancer in the patient. An effective amount of the therapeutic compound necessary to achieve a therapeutic effect may vary according to factors such as the activity of the particular compound used; time of administration; the rate of excretion of the compound; the duration of the treatment; other drugs, compounds or materials used in combination with the compound; the state of the disease or condition, age, sex, weight, condition, general health and prior medical history of the patient being treated, and like factors well-known in the medical art. Dosage regimens may be adjusted to provide the optimum therapeutic response. For example, several divided doses may be administered daily or the dose may be proportionally reduced as indicated by the exigencies under the therapeutic situation. A non-limiting example of an effective dose range for a therapeutic compound of the present disclosure is from about 0.01 to 50 mg/kg of body weight/day. One of ordinary skill in the art would be able to study the relevant factors and determine the effective amount of the therapeutic compound without undue experimentation.

The therapeutic compound can be administered to an animal at a frequently of several times a day, or it may be administered at a lower frequency, such as once a day, once a week, once every two weeks, once a month, or even at a much lower frequency, such as once every several months or even once a year or less frequently. It should be understood that, in non-limiting examples, the amount of compound dosed daily may be administered every day, every other day, every 2 days, every 3 days, every 4 days, or every 5 days. For example, for every other day administration, a 5-mg-per-day dose may be initiated on Monday with a first subsequent 5-mg-per-day dose administered on Wednesday, a second subsequent 5-mg-per-day dose administered on Friday, and so on. The frequency of the dose is readily apparent to a person skilled in the art and depends upon a number of factors, such as, but not limited to, the type and severity of the disease being treated, and the type and age of the animal. Actual dosage levels of the active ingredients in the pharmaceutical compositions of the present disclosure may be varied so as to obtain an amount of the active ingredient that is effective to achieve the desired therapeutic response for a particular patient, composition, and mode of administration, without being toxic to the patient. A doctor, e.g., a physician or veterinarian, of ordinary skill in the art may readily determine and prescribe the effective amount of the pharmaceutical composition required. For example, the physician or veterinarian could start with a dose of the antibodies of the present disclosure used in the pharmaceutical composition which is lower than the level required to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as single doses for the patients to be treated; each unit contains a predetermined quantity of therapeutic compound which is calculated to produce the desired therapeutic effect in combination with the required pharmaceutical vehicle. The dosage unit forms of the present disclosure are determined by and directly dependent on factors (a) the unique characteristics of the therapeutic compound and the particular therapeutic effect to be achieved, and (b) the limitations inherent in the art of compounding/formulating such a therapeutic compound for the treatment of cancer in a patient.

A person skilled in the art will recognize that although more than one route can be used for administration, a particular route can provide a more immediate and more effective reaction than another route.

The administration routes for the antibody or composition of the present disclosure include inhalational, oral, nasal, rectal, parenteral, sublingual, transdermal, transmucosal (e.g., sublingual, lingual, (trans)buccal, (trans)urethral, vaginal (e.g., transvaginal and intravaginal), (trans)nasal, and (trans)rectal), intravesical, intrapulmonary, intraduodenal, intragastric, intrathecal, subcutaneous, intramuscular, intradermal, intra-arterial, intravenous, intrabronchial, inhalation, and topical administration. Suitable compositions and dosage forms include, for example, a tablet, a capsule, a cachet, a pill, a gel cap, a buccal tablet, a dispersion, a suspension, a solution, a syrup, a granule, a bead, a transdermal patch, a gel, a powder, a pellet, a slurry, a pastille, a cream, a paste, an ointment, a lotion, a plate, a suppository, a liquid spray for nasal or oral administration, a dry powder or an aerosol preparation for inhalation, and a composition and a preparation for intravesical administration. It should be understood that preparations and compositions that can be used in the present disclosure are not limited to the specific preparations and compositions described herein.

Controlled- or sustained-release preparations of a pharmaceutical composition of the present disclosure may be made using conventional technology. In some cases, the dosage forms to be used can be provided as slow- or controlled-release preparations of one or more active ingredients by using, for example, hydropropylmethyl cellulose, other polymer matrices, gels, permeable membranes, osmotic systems, multilayer coatings, microparticles, liposomes, or microspheres or a combination thereof to provide desired release profiles in varying proportions. Suitable controlled-release preparations known to those of ordinary skill in the art, including those described herein, can be readily selected for the pharmaceutical compositions of the present disclosure. Thus, single unit dosage forms suitable for oral administration, such as tablets, capsules, gel caps, and cachets, which are adapted for controlled-release are encompassed by the present disclosure.

Most controlled-release pharmaceutical products have a common goal of improving drug therapy compared with their non-controlled counterparts. Ideally, the use of an optimally designed controlled-release article in medical treatment is characterized by a minimum of drug substance being used to cure or control the condition in a minimum amount of time. Advantages of controlled-release preparations include extended activity of the drug, reduced dosage frequency, and increased patient compliance. In addition, controlled-release preparations can be used to affect the time of onset of action or other characteristics, such as blood level of the drug, and thus can affect the occurrence of side effects.

In some embodiments, the present disclosure relates to a method for treating cancer in a subject in need thereof or for stimulating or enhancing an immune response in a subject in need thereof, the method comprising administering to the subject an effective amount of the antibody or the pharmaceutical composition of the present disclosure.

In some embodiments, the present disclosure relates to a method for treating cancer in a subject in need thereof or for stimulating or enhancing an immune response in a subject in need thereof, the method comprising administering to the subject an effective amount of the antibody or the pharmaceutical composition of the present disclosure, wherein the antibody or the pharmaceutical composition of the present disclosure is administered to the subject at a dose of 0.01-mg/kg/day with an interval between 1-14 days.

In some embodiments, the present disclosure relates to the use of the antibody or the pharmaceutical composition of the present disclosure in the manufacture of a drug for treating cancer in a subject in need thereof or for stimulating or enhancing an immune response in a subject in need thereof.

In a first aspect, the present disclosure relates to an antibody specifically binding to a human DKK2 protein, wherein the antibody comprises a heavy chain variable region comprising complementarity-determining regions CDRH1, CDRH2, and CDRH3, and a light chain variable region comprising complementarity-determining regions CDRL1, CDRL2, and CDRL3, wherein:
(a) CDRH1 has an amino acid sequence as shown in SEQ ID NO: 1;
(b) CDRH2 has an amino acid sequence as shown in SEQ ID NO: 2 or SEQ ID NO: 3;
(c) CDRH3 has an amino acid sequence as shown in SEQ ID NO: 4;
(d) CDRL1 has an amino acid sequence as shown in SEQ ID NO: 5;
(e) CDRL2 has an amino acid sequence as shown in SEQ ID NO: 6; and
(f) CDRL3 has an amino acid sequence as shown in SEQ ID NO: 7.

In a second aspect, the present disclosure relates to the antibody according to the first aspect, wherein the heavy chain variable region comprises an amino acid sequence as shown in SEQ ID NO: or SEQ ID NO: 14.

In a third aspect, the present disclosure relates to the antibody according to the first or the second aspect, wherein the light chain variable region comprises an amino acid sequence as shown in SEQ ID NO: 8 or SEQ ID NO: 12.

In a fourth aspect, the present disclosure relates to the antibody according to any one of the first to the third aspects, wherein the heavy chain of the antibody comprises an amino acid sequence as shown in SEQ ID NO: 11 or SEQ ID NO: 15, or an amino acid sequence having at least 95%, at least 96%, at least 97%, at least 98%, at least 99%, or at least 99.5% sequence identity to the amino acid sequence as shown in SEQ ID NO: 11 or SEQ ID NO: 15.

In a fifth aspect, the present disclosure relates to the antibody according to any one of the first to the fourth aspects, wherein the light chain of the antibody comprises an amino acid sequence as shown in SEQ ID NO: 9 or SEQ ID NO: 13, or an amino acid sequence having at least 95%, at least 96%, at least 97%, at least 98%, at least 99%, or at least 99.5% sequence identity to the amino acid sequence as shown in SEQ ID NO: 9 or SEQ ID NO: 13.

In a sixth aspect, the present disclosure relates to the antibody according to any one of the first to the fifth aspects, wherein the heavy chain variable region and/or the light chain variable region are a part of a single-chain variable fragment scFv, an F(ab′)2 fragment, an Fab or Fab′ fragment, a bivalent antibody, a trivalent antibody, a tetravalent antibody or a monoclonal antibody.

In a seventh aspect, the present disclosure relates to a pharmaceutical composition, comprising the antibody according to any one of the first to the sixth aspects and a pharmaceutically acceptable carrier.

In an eighth aspect, the present disclosure relates to a DNA molecule encoding the heavy chain and/or light chain of the antibody according to any one of the first to the sixth aspects.

In a ninth aspect, the present disclosure relates to a method for treating cancer in a subject in need thereof or for stimulating or enhancing an immune response in a subject in need thereof, the method comprising administering to the subject an effective amount of the antibody according to any one of the first to the sixth aspects or the pharmaceutical composition according to the seventh aspect.

In a tenth aspect, the present disclosure relates to the method according to the ninth aspect, wherein the antibody according to any one of the first to the sixth aspects or the pharmaceutical composition according to the seventh aspect is administered to the subject at a dose of 0.01-50 mg/kg/day with an interval between 1-14 days.

In an eleventh aspect, the present disclosure relates to the use of the antibody according to any one of the first to the sixth aspects or the pharmaceutical composition according to the seventh aspect in the manufacture of a drug for treating cancer in a subject in need thereof or for stimulating or enhancing an immune response in a subject in need thereof.

The present disclosure will be further illustrated in the following non-limiting working examples. It should be understood that these examples are only to explain the present disclosure but not to limit materials, conditions, process parameters, etc. cited herein. Unless otherwise indicated, all proportions are calculated on the basis of weight.

### EXAMPLES

5F8 was obtained from AbMax Biotechnology Co., Ltd. M-751, M-755 and M-763 were obtained by conventional experimental operations (such as changing IgG subclasses, rehumanization, changing signal peptides, amino acid point mutations, and/or a combination of the above) known in the art for humanization of antibodies using M-747 as a parental antibody. The amino acid sequences of CDRH1, CDRH2, CDRH3, CDRL1, CDRL2, CDRL3, VH (a heavy chain variable region), VL (a light chain variable region), HC (a full-length heavy chain) and LC (a full-length light chain) of 5F8, M-747, M-751, M-755 and M-763, and their corresponding SEQ ID NOs, are as shown in Table 1.

TABLE 1

| Name | Region | SEQ ID NO | Sequence |
|---|---|---|---|
| 5F8 | CDRH1 | 1 | TNYWMN |
| | CDRH2 | 16 | MIHPSDSETRLNQKFKD |
| | CDRH3 | 4 | EGRLGLRSYAMDY |
| | CDRL1 | 18 | KSSQSLLNSSNQKNYLA |
| | CDRL2 | 6 | FASTRES |
| | CDRL3 | 7 | QQHYITPLT |
| | VH | 19 | GAELVRPGASVKLSCKASGYSFTNYWMNWVKQRPGQGLEWIGMIHPSD |
| | | | SETRLNQKFKDKATLTVDKSSSTAYMQLSSPTSEDSAVYYCAREGRLG |
| | | | LRSYAMDYWGQGTSVTVSS |
| | VL | 20 | PSSLAMSVGQKVTMSCKSSQSLLNSSNQKNYLAWYQQKPGQSPKLLVY |
| | | | FASTRESGVPDRFVGSGSGTDFTLTITSVQAEDLADYFCQQHYITPLT |
| | | | FGAGTKLE |
| M-747 | CDRH1 | 1 | TNYWMN |
| | CDRH2 | 16 | MIHPSDSETRLNQKFKD |
| | CDRH3 | 4 | EGRLGLRSYAMDY |
| | CDRL1 | 18 | KSSQSLLNSSNQKNYLA |
| | CDRL2 | 6 | FASTRES |
| | CDRL3 | 7 | QQHYITPLT |
| | VH | 21 | QVQLVQSGSELKKPGASVKVSCKASGYTFTNYWMNWVRQAPGQGLEWM |
| | | | GMIHPSDSETRLNQKFKDRVTITVDKSTSTAYMELSSLRSEDTAVYYC |
| | | | AREGRLGLRSYAMDYWGQGTLVTVSS |
| | VL | 22 | DIVMTQSPDSLAVSLGERATINCKSSQSLLNSSNQKNYLAWYQQKPGQ |
| | | | PPKLLVYFASTRESGVPDRFSGSGSGTDFTLTISSLQAEDVAVYFCQQ |
| | | | HYITPLTFGGGTKVEIKR |
| | HC | 23 | MEWSWVFLFFLSVTTGVHSQVQLVQSGSELKKPGASVKVSCKASGYTF |
| | | | TNYWMNWVRQAPGQGLEWMGMIHPSDSETRLNQKFKDRVTITVDKSTS |
| | | | TAYMELSSLRSEDTAVYYCAREGRLGLRSYAMDYWGQGTLVTVSSAST |
| | | | KGPSVFPLAPSSKSTSGGTAALGCLVKDYFPEPVTVSWNSGALTSGVH |
| | | | TFPAVLQSSGLYSLSSVVTVPSSSLGTQTYICNVNHKPSNTKVDKRVE |
| | | | PKSCDKTHTCPPCPAPELLGGPSVFLFPPKPKDTLMISRTPEVTCVVV |
| | | | DVSHEDPEVKFNWYVDGVEVHNAKTKPREEQYNSTYRVVSVLTVLHQD |
| | | | WLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYS |
| | | | KLTVDKSRWQQGNVFSCSVMHEALHNHYTQKSLSLSPGK |
| | LC | 24 | MDMRVPAQLLGLLLLWLRGARCDIVMTQSPDSLAVSLGERATINCKSS |
| | | | QSLLNSSNQKNYLAWYQQKPGQPPKLLVYFASTRESGVPDRFSGSGSG |
| | | | TDFTLTISSLQAEDVAVYFCQQHYITPLTFGGGTKVEIKRTVAAPSVF |
| | | | IFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRG |
| | | | EC |
| M-75 | CDRH1 | 1 | TNYWMN |
| | CDRH2 | 2 | MIHPSDSETRLNQKFQG |
| | CDRH3 | 4 | EGRLGLRSYAMDY |
| | CDRL1 | 5 | RSSQSLLNSSNQKNYLA |
| | CDRL2 | 6 | FASTRES |
| | CDRL3 | 7 | QQHYITPLT |
| | VH | 10 | QVQLVQSGAEVKKPGSSVKVSCKASGYTFTNYWMNWVRQAPGQGLEWM |
| | | | GMIHPSDSETRLNQKFQGRVTITVDKSTSTAYMELSSLRSEDTAVYYC |
| | | | AREGRLGLRSYAMDYWGQGTLVTVSS |
| | VL | 8 | DIQMTQSPSSLSASVGDRVTITCRSSQSLLNSSNQKNYLAWYQQKPGK |
| | | | VPKLLIYFASTRESGVPSRFSGSGSGTDFTLTISSLQPEDVATYFCQQ |
| | | | HYITPLTFGGGTKVEIKR |
| | HC | 11 | MDMRVPAQLLGLLLLWLRGARCQVQLVQSGAEVKKPGSSVKVSCKASG |
| | | | YTFTNYWMNWVRQAPGQGLEWMGMIHPSDSETRLNQKFQGRVTITVDK |
| | | | STSTAYMELSSLRSEDTAVYYCAREGRLGLRSYAMDYWGQGTLVTVSS |
| | | | ASTKGPSVFPLAPCSRSTSESTAALGCLVKDYFPEPVTVSWNSGALTS |
| | | | GVHTFPAVLQSSGLYSLSSVVTVPSSSLGTKTYTCNVDHKPSNTKVDK |
| | | | RVESKYGPPCPPCPAPEFLGGPSVFLFPPKPKDTLMISRTPEVTCVVV |
| | | | DVSQEDPEVQFNWYVDGVEVHNAKTKPREEQFNSTYRVVSVLTVLHQD |
| | | | WLNGKEYKCKVSNKGLPSSIEKTISKAKGQPREPQVYTLPPSQEEMTK |
| | | | NQVSLTCLVKGFYPSDIAVEWESNGQPENNYKTTPPVLDSDGSFFLYS |
| | | | RLTVDKSRWQEGNVFSCSVMHEALHNHYTQKSLSLSLGK |
| | LC | 9 | MDMRVPAQLLGLLLLWLRGARCDIQMTQSPSSLSASVGDRVTITCRSS |
| | | | QSLLNSSNQKNYLAWYQQKPGKVPKLLIYFASTRESGVPSRFSGSGSG |
| | | | TDFTLTISSLQPEDVATYFCQQHYITPLTFGGGTKVEIKRTVAAPSVF |
| | | | IFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRG |
| | | | EC |
| M-755 | CDRH1 | 1 | TNYWMN |
| | CDRH2 | 3 | MIHPSDSETRLNQKLQG |
| | CDRH3 | 4 | EGRLGLRSYAMDY |
| | CDRL1 | 5 | RSSQSLLNSSNQKNYLA |
| | CDRL2 | 6 | FASTRES |
| | CDRL3 | 7 | QQHYITPLT |
| | VH | 14 | QVQLVQSGAEVKKPGASVKVSCKASGYTFTNYWMNWVRQAPGQGLEWM |
| | | | GMIHPSDSETRLNQKLQGRVTITVDKSTSTAYMELRSLRSDDTAVYYC |
| | | | AREGRLGLRSYAMDYWGQGTLVTVSS |
| | VL | 12 | DIQMTQSPSSLSASVGDRVTITCRSSQSLLNSSNQKNYLAWYQQKPGK |
| | | | APKLLIYFASTRESGVPSRFSGSGSGTDFTLTISSLQPEDFATYFCQQ |
| | | | HYITPLTFGGGTKVEIKR |
| | HC | 15 | MDMRVPAQLLGLLLLWLRGARCQVQLVQSGAEVKKPGASVKVSCKASG |
| | | | YTFTNYWMNWVRQAPGQGLEWMGMIHPSDSETRLNQKLQGRVTITVDK |
| | | | STSTAYMELRSLRSDDTAVYYCAREGRLGLRSYAMDYWGQGTLVTVSS |
| | | | ASTKGPSVFPLAPCSRSTSESTAALGCLVKDYFPEPVTVSWNSGALTS |
| | | | GVHTFPAVLQSSGLYSLSSVVTVPSSSLGTKTYTCNVDHKPSNTKVDK |
| | | | RVESKYGPPCPPCPAPEFLGGPSVFLFPPKPKDTLMISRTPEVTCVVV |
| | | | DVSQEDPEVQFNWYVDGVEVHNAKTKPREEQFNSTYRVVSVLTVLHQD |
| | | | WLNGKEYKCKVSNKGLPSSIEKTISKAKGQPREPQVYTLPPSQEEMTK |
| | | | NQVSLTCLVKGFYPSDIAVEWESNGQPENNYKTTPPVLDSDGSFFLYS |
| | | | RLTVDKSRWQEGNVFSCSVMHEALHNHYTQKSLSLSLGK |
| | LC | 13 | MDMRVPAQLLGLLLLWLRGARCDIQMTQSPSSLSASVGDRVTITCRSS |
| | | | QSLLNSSNQKNYLAWYQQKPGKAPKLLIYFASTRESGVPSRFSGSGSG |
| | | | TDFTLTISSLQPEDFATYFCQQHYITPLTFGGGTKVEIKRTVAAPSVF |
| | | | IFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRG |
| | | | EC |
| M-763 | CDRH1 | 1 | TNYWMN |
| | CDRH2 | 16 | MIHPSDSETRLNQKFKD |
| | CDRH3 | 4 | EGRLGLRSYAMDY |
| | CDRL1 | 17 | KSSQSLLQSSNQKNYLA |
| | CDRL2 | 6 | FASTRES |
| | CDRL3 | 7 | QQHYITPLT |
| | VH | 25 | QVQLVQSGAELKKPGASVKVSCKASGYTFTNYWMNWVRQAPGQGLEWM |
| | | | GMIHPSDSETRLNQKFKDRVTITVDKSTSTAYMELSSLRSEDTAVYYC |
| | | | AREGRLGLRSYAMDYWGQGTLVTVSS |
| | VL | 26 | DIVMTQSPDSLAVSLGERATINCKSSQSLLQSSNQKNYLAWYQQKPGQ |
| | | | PPKLLVYFASTRESGVPDRFSGSGSGTDFTLTISSLQAEDVAVYFCQQ |
| | | | HYITPLTFGGGTKVEIKR |
| | HC | 27 | MDMRVPAQLLGLLLLWLRGARCQVQLVQSGAELKKPGASVKVSCKASG |
| | | | YTFTNYWMNWVRQAPGQGLEWMGMIHPSDSETRLNQKFKDRVTITVDK |
| | | | STSTAYMELSSLRSEDTAVYYCAREGRLGLRSYAMDYWGQGTLVTVSS |
| | | | ASTKGPSVFPLAPCSRSTSESTAALGCLVKDYFPEPVTVSWNSGALTS |
| | | | GVHTFPAVLQSSGLYSLSSVVTVPSSSLGTKTYTCNVDHKPSNTKVDK |
| | | | RVESKYGPPCPPCPAPEFLGGPSVFLFPPKPKDTLMISRTPEVTCVVV |
| | | | DVSQEDPEVQFNWYVDGVEVHNAKTKPREEQFNSTYRVVSVLTVLHQD |
| | | | WLNGKEYKCKVSNKGLPSSIEKTISKAKGQPREPQVYTLPPSQEEMTK |
| | | | NQVSLTCLVKGFYPSDIAVEWESNGQPENNYKTTPPVLDSDGSFFLYS |
| | | | RLTVDKSRWQEGNVFSCSVMHEALHNHYTQKSLSLSLGK |
| | LC | 28 | MDMRVPAQLLGLLLLWLRGARCDIVMTQSPDSLAVSLGERATINCKSS |
| | | | QSLLQSSNQKNYLAWYQQKPGQPPKLLVYFASTRESGVPDRFSGSGSG |
| | | | TDFTLTISSLQAEDVAVYFCQQHYITPLTFGGGTKVEIKRTVAAPSVF |
| | | | IFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRG |
| | | | EC |

### EXAMPLE 1: Preparation of Antibody

The antibody materials were produced from Chinese hamster ovary (CHO) suspension cell lines. Basically, as described by ThermoFisher (catalog number A29133, file part number A29518), 5 the ExpiCHO-STM “maximum titer” method was followed. The pcDNA3.4 expression vector containing a LC or a HC coding region was co-transfected into CHO-S cells at VCD of 6×10⁶ using ExpiFectamine. Under the conditions of 32° C., 5% CO2, and 85% humidity, the expression lasted for days at a shaking speed of 500 RPM (3 mm orbit) or 130 RPM (19 mm orbit). All clear supernatants were produced by precipitating the cells at 3000 g for 20 minutes and then performing μm filtration. The antibody was purified from the clear supernatant using Mab Select SuRe protein A resin. A sodium phosphate and sodium chloride buffer system with arginine washing and pH 3.5 acetate elution was used. The protein A eluent was neutralized with tris, and the buffer was exchanged to 20 mM sodium phosphate, 150 mM NaCl, pH 7.4.

### EXAMPLE 2: Binding Affinity Test

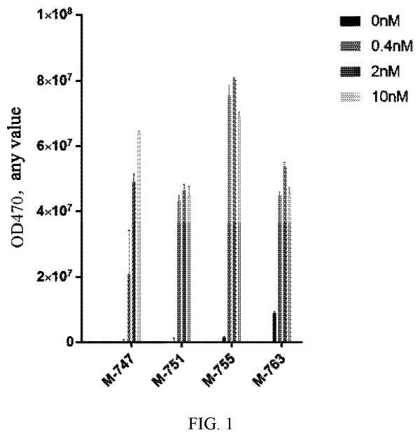

Enzyme-linked immunosorbent assay (ELISA) was used to assess the binding affinity of candidate antibodies (M-751, M-755 and M-763) for a human DKK2 protein compared with the parental antibody (M-747). The human DKK2 protein (R & D system, catalog number: 6628-DK-010) was coated in 384-well plates at 1 ng protein/well in a 50 ul 1× PBS buffer. The coated plates were sealed and incubated at 4° C. overnight. Then the plates were washed twice with 1× PBS buffer at 60 ul/well, and then blocked with 3% BSA-PBST at 50 ul/well, sealed and incubated at 37° C. for 2 hours. After washing twice, 50 ul of the primary antibody which was diluted with 1% BSA-PBST to a concentration as shown in the figure was added to each well of the plates, and then incubated at 37° C. for 2 hours. After washing twice, 50 ul of the secondary antibody (goat anti-human IgG-Fc HRP-labelled antibody) (Bethyl Laboratories, catalog number: A80-104P) which was diluted with 1% BSA-PBST at 1: 5000 was added to each well of the plates and incubated at 37° C. for 1 hour. After washing twice, 80 ul of SuperSignal™ West Pico PLUS chemiluminescent substrate (Thermo Scientific, catalog number: 34580) was added to each well at room temperature. Plates were read on the EnVision multimode plate reader at 470 nm. The assay was repeated three rounds. Each variant was tested in triplicate in each round. FIG. 1 shows the representative data from one of the three rounds. Bars represent mean±SEM.

### EXAMPLE 3: Assessment of Conformational Stability of Candidate Antibodies by DSF Using Materials Produced by Stable Cell Lines

Production of antibodies using the CHO stable transfection system: Using Lipofectamine 3000 (Invitrogen L3000-015), the expression plasmid pDL5 (GenScript) having an antibody heavy chain coding sequence and the expression plasmid pDL2 (GenScript) having an antibody light chain coding sequence as well as the helper plasmid pDL4 were co-transfected into CHO-K 1 host cells according to the manufacturer's recommended procedure. The transfected cells were incubated in a medium without selective pressure for 1 day and then incubated in a medium containing 20 mg/L puromycin for 6 days. The recovered cells were cultured in production medium to allow the secreted antibodies to accumulate in the spent media.

Small-scale purification: Using Protein-A column (Nab Protein A Plus Spin Column, Thermo Scientific, catalog number 89952), the spent media were subjected to antibody purification after centrifugation and clarification according to the manufacturer's procedure. The purified antibody was then subjected to endotoxin removal by a high-capacity endotoxin removal column (Thermo Scientific, catalog number 88274). The endotoxin level after the removal step was tested using the E-Toxate kit (Sigma-Aldrich, catalog numbers ET0100, ET0200 and ET0300) and confirmed to be lower than 0.1 EU/mL. The antibody concentration was measured at OD280.

DSF assessment: The purified candidate antibodies were subjected to differential scanning fluorescence (DSF) analysis to determine its relative conformational stability (Table 2). Compared with the parental molecule M-747, all candidate antibodies showed improved stability.

TABLE 2
DSF for candidate antibodies compared with the parental antibody.
Higher WSS values indicate better thermal stability.

| Molecule | WSS rep 1 | WSS rep 2 | T1 Rep 1 | T1 Rep 2 | T2 Rep 1 | T2 Rep 2 |
|---|---|---|---|---|---|---|
| M-747 | 6.7 | 6.9 | 71.2 | 70.8 | — | 72.4 |
| M-751 | 55.1 | 52.8 | 67.3 | 67.1 | 84.6 | 84.6 |
| M-755 | 42.8 | 41.7 | 67.3 | 67.5 | 80.9 | 80.8 |
| M-763 | 22.8 | 22.6 | 67.3 | 67.3 | 84.6 | 84.6 |

### EXAMPLE 4: Assessment of Efficacy of Candidate Antibodies in Animal Models Using Materials Produced by Stable Cell Lines

The in vivo activities of candidate antibodies produced by stable cell lines (see Example 3, the production and purification sections) were assessed in C57BL/6 female mice (Envigo) using syngeneic tumor models. Multiclonal human IgG (BioXCell, catalog number BE0092) was used as a negative control. 5F8 was used as a positive control. The effects of the antibodies obtained above on tumor growth and immune cell activation were compared with those in the positive group and the negative group. Compared with the parental molecule M-747, M-751 and M-755 showed stronger tumor suppression and immune activation effects.

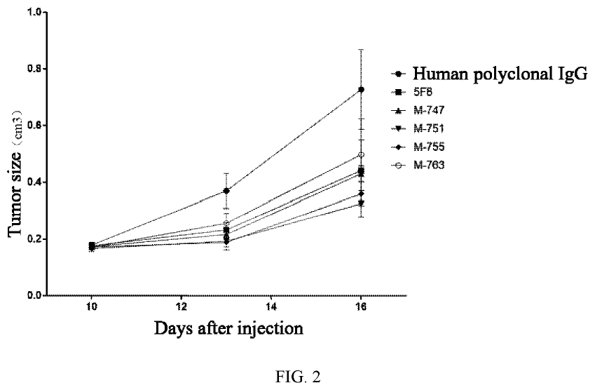

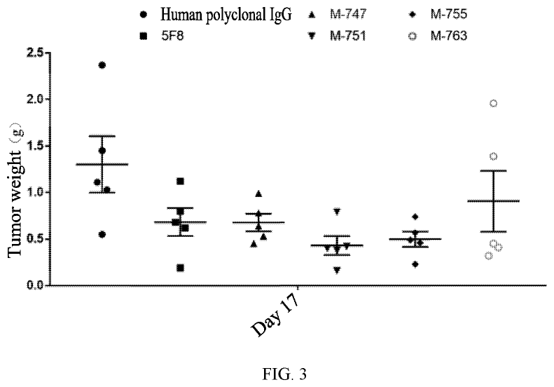

Tumor transplantation: The mouse colon cancer cell line MC38 (Kerafast, catalog number ENH204) was inoculated subcutaneously in the right axillary region (lateral) of the mouse. Tumor volumes were monitored in two dimensions using a caliper, and the volume was expressed in mm 3 using the formula: V=0.5 a×b 2, where a and b are the long and short diameters of the tumor, respectively. Then on the 10th day after inoculation, when the average tumor volume reached 200 mm 3, the animals were randomly divided into groups (n=5) for treatment. On day 10, day 13 and day 16, anti-human DKK2 antibodies were injected intraperitoneally (IP) at 12.5 mg/kg (FIG. 2). On day 17, the mice were sacrificed and the tumors were weighed (FIG. 3).

FIG. 2 shows tumor volumes measured on day 10, day 13 and day 16 after injection in groups treated with a candidate antibody (M-751, M-755 or M-763), a parental antibody (M-747) or a control (human polyclonal IgG or 5F8). The results were expressed as mean±SEM. By comparing the average tumor sizes, M-751 treatment and M-755 treatment showed stronger tumor growth control than M-747.

FIG. 3 shows mean endpoint tumor weights on day 17 after injection in groups treated with a candidate antibody (M-751, M-755 or M-763), a parental antibody (M-747) or a control (human polyclonal IgG or 5F8). The results were expressed as mean±SEM. By comparing the average tumor weights, M-751 treatment and M-755 treatment showed stronger tumor growth control than M-747.

Preparation of tumor infiltrating leukocytes: The collected tumors were minced with scissors and scalpel blades and incubated with a digestive buffer (RPMI 1640 medium, 5% FBS, 1% penicillin/streptomycin, 25 mM HEPES and 300 U collagenase (Sigma C0130)) on a shaker at 37° C. for 2 hours. The dispersed cells were filtered through a 70 micron cell filter to remove masses and debris. After centrifugation at 500 g and 4° C. for 5 minutes, the cell pellets were resuspended in a red blood cell lysing buffer (Sigma R7757) and incubated at room temperature for 5 minutes to remove red blood cells. The cells were pelleted again, resuspended, incubated at 37° C. in 0.05% trypsin/EDTA for 5 minutes, and then digested DNA with 1 μg/ml type I DNase (Sigma, D4263) for minutes. Trypsin digestion was stopped by adding FBS to 5% and the cells were filtered again through a 40-μm cell filter. Finally, the cells were pelleted again and resuspended in PBS at a concentration of 2×10⁷.

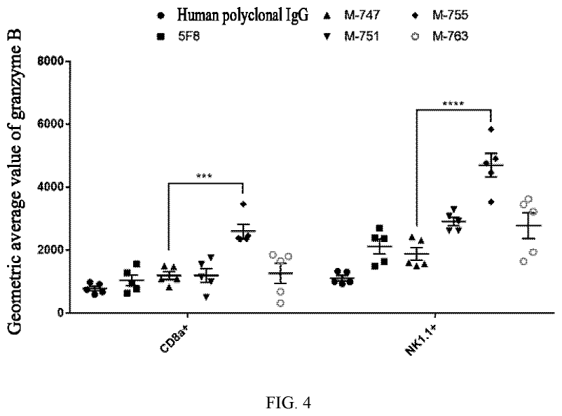

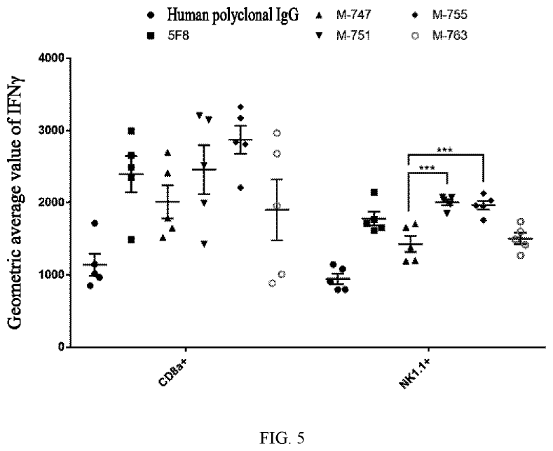

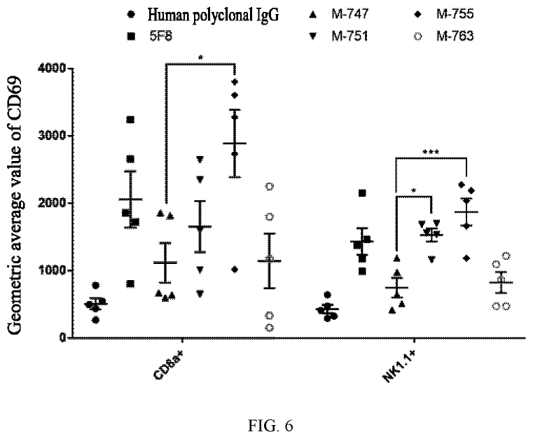

Flow cytometry: Cells in a single-cell suspension were fixed with 2% PFA (Santa Cruz, sc-281692). After washing with a flow cytometry staining buffer (eBioscience, 00-4222-26), the cells were stained with cell surface marker antibodies on ice in the dark for 1 hour. To stain intracellular proteins, the cells were washed and resuspended in a permeabilization buffer (BD, 554723), and then stained with antibodies in a permeabilization buffer for 1 h on ice in the dark. Then the cell pellets were pelleted and resuspended in a flow cytometry staining buffer for flow cytometry analysis. FIG. 4, FIG. 5 and FIG. 6 show the geometric average values of granzyme B, IFNγ and CD69 from different treatment groups. Data were expressed as mean±SEM. Statistical significance was analyzed by one-way analysis of variance. *: p<0.05; ***: P<0.0005; ****: p<0.0001.

### EXAMPLE 5: Comparison of Developability of M-751, M-755 and the Parental Molecule when Using Materials Produced by Fed-Batch Production

Fed-batch production: The stable pools of M-747, M-751, and M-755 were thawed and expanded to produce more representative materials (compared to transient (example 2) and laboratory-scale materials (example 3 and example 4)) in a fed-batch setup for non-clinical and clinical development for further analysis. The stable pool from each molecule was expanded to a shake flask and passaged in the medium M1 (CD FortiCHO+4 mM glutamine+5 μg/mL blasticidin+10 μg/mL puromycin) for about 2 weeks, and then inoculated for fed-batch culture. After fed-batch inoculation, the cells were directly diluted into the medium M2 (ActiPro+4 mM glutamine+1X GS replenisher) at a density of about 10×10 6 cells/mL in shake flasks. The initial working volume on day 0 was 200 mL. The fed-batch cultures were incubated in an InFors HT incubator (37.0° C., 85% humidity, 5% CO2, 110 RPM, 50 mm orbital motion). The feed culture medium was added to the fed-batch cultures on days 1, 3, 5, 7 and 9. Glucose was added to the culture to maintain its level of >3 g/L, and GlutaMAX was added to the culture to maintain the glutamine level of >2 mM. On day 2, when the cell density reached about 20×10 6 cells/mL, the culture temperature was adjusted to 32° C. The fed-batch cultures were harvested on day 10. Titers of supernatants from harvested cultures were measured by a Cedex Bio analyzer. The supernatants of fed-batch cultures were purified by one-step protein A chromatography.

SEC analysis: Samples were tested in the following manner: directly in the form of protein A eluate without adjusting the pH, or using 1 M sodium acetate to neutralize samples to pH 5.5, or under conditions of maintaining low pH for the period as shown in the table, using 1 M acetic acid to adjust samples to pH 3.7 (Table 3). The samples were diluted to 10 mg/mL using a formulated buffer (20 mM histidine, 250 mM sorbitol and 0.2% poloxamer 188) for injection. The column was washed with Milli-Q water at a flow rate of 0.5 mL/min for no less than 60 minutes before starting the sample setup, then the mobile phase was used for equilibrating the system until a stable baseline and back pressure are reached or for at least 40 minutes. Each sample was injected twice. After the sample was injected up to ten times, the reference standard in bracket was injected. Empower was used to integrate chromatograms. The % main peak, % HMW (high molecular weight) and % LMW (low molecular weight) were calculated as follows:
% HMW=(sum of HMW peak area)/(total peak area)*100%
% main peak=(main peak area)/(total peak area)*100%
% LMW=(sum of LMW peak area)/(total peak area)*100%
Data were expressed as the average of two injections.

TABLE 3
Stability assessment of M-751, M-755 and M-747. Samples
were analyzed by SEC under various treatment conditions.
Compared with the parental molecule M-747, both M-751
and M-755 showed smaller % HMW under all conditions.

| | | | |
|---|---|---|---|
| Molecule | % HMW | % Main peak | % LMW |

Without adjusting

| | | | |
|---|---|---|---|
| M-747 | 16.52 | 83.07 | 0.41 |
| M-751 | 2.94 | 96.67 | 0.39 |
| M-755 | 4.23 | 95.08 | 0.69 |

pH adjusted to 5.5

| | | | |
|---|---|---|---|
| M-747 | 16.68 | 82.99 | 0.33 |
| M-751 | 1.68 | 98.31 | 0.01 |
| M-755 | 3.87 | 96.08 | 0.05 |

Low pH (pH 3.7) for 1 hour

| | | | |
|---|---|---|---|
| M-747 | 16.3 | 83.5 | 0.3 |
| M-751 | 2.9 | 98.1 | 0.1 |
| M-755 | 4.9 | 94.9 | 0.1 |

Low pH (pH 3.7) for 2 hours

| | | | |
|---|---|---|---|
| M-747 | 15.6 | 84.1 | 0.3 |
| M-751 | 3.7 | 96.0 | 0.3 |
| M-755 | 5.5 | 94.3 | 0.2 |

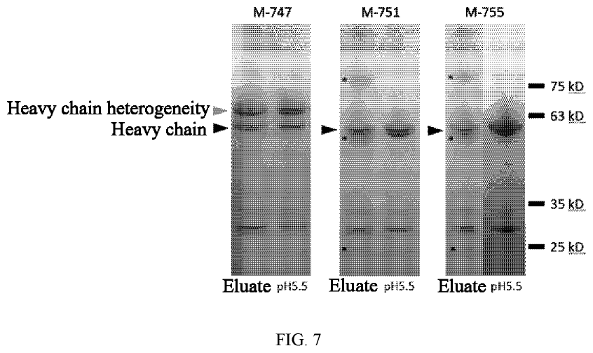

SDS-PAGE analysis: Samples were tested in the following manner: directly in the form of protein A eluate without adjusting the pH, or using 1 M sodium acetate to neutralize samples to pH Before loading, the samples were diluted with Milli-Q water to 1 mg/mL, mixed with the loading buffer (EZ Biolab, LS003) at 4: 1. The mixture was incubated at 100° C. for 10 minutes, and then centrifuged at 10,000 rpm for 5 minutes. Pre-stained protein markers (Tanon, 180-6003) and samples were loaded into gels (Tanon, 180-8008) at 5 μl/well. Electrophoresis was run at 80 V for 10 minutes, and then at 160 V until the dye front run off the gel. The gel was then stained with a staining solution (Tanon, 180-7001) for 60 minutes, washed three times with Milli-Q water, and then imaged (FIG. 7). The parental molecule M-747 showed heavy chain heterogeneity, whereas M-751 and M-755 (sequence optimized) showed typical IgG profiles. Some heavy and light chain fragments (represented by *) were observed in M-751 and M-755 eluates, but decreased after pH adjustment.

Self-association binding constant: At high concentrations, undesirable solution properties such as self-association can pose significant challenges to preparation development. In addition to potential manufacturing and delivery challenges, self-associating substances can also affect biological activity and pharmacokinetic properties. The self-association binding constants of M-751 and M-755 were measured compared with the parental molecule M-747. The results show that M-751 and M-755 were similar to each other, and their performance was better than that of M-747 (Table 4).

The self-association binding constants were measured using the forteBIO Octet technique (Octet RED96). The antibodies were diluted to 150 ug/mL with an acetate buffer (pH 5.5), and then added to sample plates (forteBIO, Greiner, PN655209). A ProA biosensor (forteBIO) was used to capture antibodies. Measurements were performed using basic dynamics under the following settings: baseline 60/load 900/baseline 60/association 300/dissociation 300. The association (Kon) and dissociation (Kdis) rate constants of the target antigen were calculated and used to obtain the dissociation constant (KD). All calculations were performed using software provided by forteBIO.

TABLE 4
The self-association binding constants of M-751, M-755 and M-
747 were calculated. Both M-751 and M-755 showed lower self-
association affinities, whereas the parental molecule M-747
showed a higher tendency to form a stable self-aggregate.

| Variant | Self-association constant (KD, mM) in acetate at pH 5.5 |
|---|---|
| M-751 | 0.438 ± 0.086 |
| M-755 | 0.456 ± 0.101 |
| M-747 | 0.208 ± 0.040 |

Although the specific embodiments have been described, for the applicant or a person skilled in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents.

1. An antibody specifically binding to a human DKK2 protein, wherein the antibody comprises a heavy chain variable region comprising complementarity-determining regions CDRH1, CDRH2, and CDRH3, and a light chain variable region comprising complementarity-determining regions CDRL1, CDRL2, and CDRL3, wherein:
(a) CDRH1 has an amino acid sequence as shown in SEQ ID NO: 1;
(b) CDRH2 has an amino acid sequence as shown in SEQ ID NO: 2 or SEQ ID NO: 3;
(c) CDRH3 has an amino acid sequence as shown in SEQ ID NO: 4;
(d) CDRL1 has an amino acid sequence as shown in SEQ ID NO: 5;
(e) CDRL2 has an amino acid sequence as shown in SEQ ID NO: 6; and
(f) CDRL3 has an amino acid sequence as shown in SEQ ID NO: 7.

2. The antibody according to claim 1, wherein the heavy chain variable region comprises an amino acid sequence as shown in SEQ ID NO: 10 or SEQ ID NO: 14.

3. The antibody according to claim 2, wherein the light chain variable region comprises an amino acid sequence as shown in SEQ ID NO: 8 or SEQ ID NO: 12.

4. The antibody according to claim 3, wherein the heavy chain of the antibody comprises an amino acid sequence as shown in SEQ ID NO: 11 or SEQ ID NO: 15, or an amino acid sequence having at least 95%, at least 96%, at least 97%, at least 98%, at least 99%, or at least 99.5% sequence identity to the amino acid sequence as shown in SEQ ID NO: 11 or SEQ ID NO: 15.

5. The antibody according to claim 4, wherein the light chain of the antibody comprises an amino acid sequence as shown in SEQ ID NO: 9 or SEQ ID NO: 13, or an amino acid sequence having at least 95%, at least 96%, at least 97%, at least 98%, at least 99%, or at least 99.5% sequence identity to the amino acid sequence as shown in SEQ ID NO: 9 or SEQ ID NO: 13.

6. The antibody according to claim 1,
wherein the heavy chain variable region and/or the light chain variable region are a part of a single-chain variable fragment scFv, an F(ab′)2 fragment, an Fab or Fab′ fragment, a bivalent antibody, a trivalent antibody, a tetravalent antibody or a monoclonal antibody.

7. A pharmaceutical composition, comprising the antibody according to claim 1 and a pharmaceutically acceptable carrier.

8. A DNA molecule, encoding the heavy chain and/or light chain of the antibody according to claim 1.

9. A method for treating cancer in a subject in need thereof or for stimulating or enhancing an immune response in a subject in need thereof, the method comprising administering to the subject an effective amount of the antibody according to claim 1.

10. The method according to claim 9, wherein the antibody is administered to the subject at a dose of 0.01-50 mg/kg/day with an interval between 1-14 days.

11. Use of the antibody according to claim 1 in the manufacture of a drug for treating cancer in a subject in need thereof or for stimulating or enhancing an immune response in a subject in need thereof.
